(19) United States
(12) Patent Application Publication
(10) Pub. No.: US 2006/0135443 A1
(43) Pub. Date: Jun. 22, 2006

(54) Use of Na*/K*-ATPase inhibitors and antagonists thereof

(75) Inventors: Mehran Khodadoust, Brookline, MA (US); Ajay Sharma, Sudbury, MA (US)

Correspondence Address: FISH & NEAVE IP GROUP;ROPES & GRAY LLP, ONE INTERNATIONAL PLACE, BOSTON, MA, 02110-2624, US

(73) Assignee: Bionaut Pharmaceuticals, Inc., Cambridge, MA (US)

(21) Appl. No.: 11/254,246

(22) Filed: Oct. 18, 2005

Related U.S. Application Data
(60) Provisional application No. 60/619,637, filed on Oct. 18, 2004.

(51) Int. Cl. A61K 31/58; A61K 31/704

(52) U.S. Cl. 514/26; 514/172

(57) ABSTRACT

The reagent, pharmaceutical formulation, kit, and methods of the invention provides a new approach for treating hypoxia-related pathological conditions, such as Alzheimer's Disease, and those involving excessive angiogenesis, especially those non-cancer pathological conditions. The invention provides the use of Na⁺/K⁺-ATPase inhibitors, such as cardiac glycosides (e.g. ouabain and proscillaridin, etc.), either alone or in combination with other standard therapeutic agents for treating such conditions. The invention also relates to the use of cardiac glycoside inhibitors/antagonists as reagents, pharmaceutical formulations, or in kits and methods for treating conditions arising from excessive amount of cardiac glycosides, including all symptoms of digitalis poisoning, depression, hypertension, etc. The pharmaceutical formulation of the invention may be delivered to a patient either systemically or locally, or both. The pharmaceutical formulations of the invention may be delivered either in one dose, or continuously over a sustained period of time using, for example, sustained drug delivery devices.

### REFERENCE TO RELATED APPLICATION

This application claims the benefit of the filing date of U.S. Provisional Application Ser. No. 60/619,637, entitled “USE OF Na⁺/K⁺-ATPASE INHIBITORS AND ANTAGONISTS THEREOF,” and filed on Oct. 18, 2004. The teachings of the referenced application are incorporated herein by reference.

### BACKGROUND OF THE INVENTION

Angiogenesis is driven by a balance between different positive and negative effector molecules influencing the growth rate of capillaries. Various angiogenetic and anti-angiogenetic factors have been cloned to date and are known (Leung et al., Science 246: 1306-9, 1989; Ueno et al., Biochem Biophys Acta 1382: 17-22, 1998; Miyazono et al., Prog Growth Factor Res. 3: 207-17, 1991). Vascular endothelial growth factor (VEGF) and trombospondin-1 (TSP-1) are two of the most well studied. VEGF is an angiogenic factor as opposed to TSP-1, which functions as an anti-angiogenic molecule (Tuszynski et al., Bioessays 18: 71-6, 1996; Dameron, et al. Science 265: 1582-4, 1994). Normal vessel growth results by balanced and coordinated expression of these opposing factors. A switch from normal to uncontrolled vessel growth can occur by up-regulating angiogenesis stimulators or down-regulating angiogenesis inhibitors, suggesting that the angiogenetic process is tightly regulated by the oscillation between these opposing forces (Bouck et al., Adv Cancer Res. 69: 135-74, 1996). For example, in tumor tissues the switch to an angiogenic phenotype occurs as a distinct step before progression to a neoplastic phenotype and is linked to epigenetic or genetic changes (Hanahan et al., Cell 86: 353-64, 1996). In support of this theory, mRNA expression of VEGF is up-regulated in aggressive tumor cell lines expressing an activated ras oncogene (Rak et al., Neoplasia 1: 23-30, 1999). Conversely, transcription of VEGF is down-regulated in these same tumor cell lines after disruption of the mutant ras allele, thus eliminating VEGF expression and rendering the cells incapable of tumor formation in vivo (Stiegler et al., J Cell Physiol. 179: 233-6, 1999). The switch to an angiogenic phenotype has also been associated with the inactivation of the tumor suppressor gene p53 (Holmgren et al., Oncogene 17: 819-24, 1998). Conversely, cell lines that are p16 deleted revert to an anti-angiogenic phenotype upon the restoration of wild type cyclin dependent kinase (cdk) inhibitor p.sup.16. Harada et al., Cancer Research 59: 3783-3789, 1999.

Besides tumors, VEGF also has been reported to cause pathological angiogenesis and this contributes to conditions such as diabetic retinopathy, rheumatoid arthritis, choroidal neovascularization, syogenic granuloma, endometriosis, pulmonary edema, and pulmonary tuberculosis.

HIF-1 is a transcription factor and is critical to survival in hypoxic conditions, both in cancer and cardiac cells. HIF-1 is composed of the O₂— and growth factor-regulated subunit HIF-1, and the constitutively expressed HIF-1 subunit (arylhydrocarbon receptor nuclear translocator, ARNT), both of which belong to the basic helix-loop-helix (bHLH)-PAS (PER, ARNT, SIM) protein family. So far in the human genome 3 isoforms of the subunit of the transcription factor HIF have been identified: HIF-1, HIF-2 (also referred to as EPAS-1, MOP2, HLF, and HRF), and HIF-3 (of which HIF-32 also referred to as IPAS, inhibitory PAS domain).

Under normoxic conditions, HIF-1 is targeted to ubiquitinylation by pVHL and is rapidly degraded by the proteasome. This is triggered through posttranslational HIF-hydroxylation on specific proline residues (proline 402 and 564 in human HIF-1 protein) within the oxygen dependent degradation domain (ODDD), by specific HIF-prolyl hydroxylases (HPH1-3 also referred to as PHD1-3) in the presence of iron, oxygen, and 2-oxoglutarate. The hydroxylated protein is then recognized by pVHL, which functions as an E3 ubiquitin ligase. The interaction between HIF-1 and pVHL is further accelerated by acetylation of lysine residue 532 through an N-acetyltransferase (ARD1). Concurrently, hydroxylation of the asparagine residue 803 within the C-TAD also occurs by an asparaginyl hydroxylase (also referred to as FIH-1), which by its turn does not allow the coactivator p300/CBP to bind to HIF-1 subunit. In hypoxia HIF-1 remains not hydroxylated and stays away from interaction with pVHL and CBP/p300 (FIG. 1). Following hypoxic stabilization HIF-1 translocates to the nucleus where it heterodimerizes with HIF-1. The resulting activated HIF-i drives the transcription of over 60 genes important for adaptation and survival under hypoxia including glycolytic enzymes, glucose transporters Glut-1 and Glut-3, endothelin-1 (ET-1), VEGF (vascular endothelial growth factor), tyrosine hydroxylase, transferrin, and erythropoietin (Brahimi-Horn et al., Trends Cell Biol 11(11): S32-S36, 2001; Beasley et al., Cancer Res 62(9): 2493-2497, 2002; Fukuda et al., J Biol Chem 277(41): 38205-38211, 2002; Maxwell and Ratcliffe, Semin Cell Dev Biol 13(1): 29-37, 2002).

Hypoxia appears to promote angiogenesis and tumor growth by promoting expression of certain angiogenesis factors such as VEGF. The inventors have discovered that certain agents in fact promote an hypoxic stress response in certain cells, such as tumor cells, which accordingly should have a direct consequence on clinical and prognostic parameters and create a therapeutic challenge. This hypoxic response includes induction of HIF-1 dependent transcription.

Hypoxia-mediated angiogenesis also plays a part in the pathogenesis of rheumatoid arthritis. For instance, intra-articular application of the angiostatic molecule angiostatin reduces the severity of collagen-induced arthritis in mice. Moreover, recent data indicate that the expression of HIF-1alpha in myeloid cells is important for the initiation of the inflammatory infiltrate in rheumatoid arthritis. See Distler, Hypoxia and angiogenesis in rheumatic diseases, Z. Rheumatol. 62(Suppl 2): 1143-5, 2003.

It has long been known that hypoxia-induced angiogenesis occurs especially in the eye, but the mechanism was unknown. Ashton et al. (Br. J. Ophthalmol. 38: 397-432, 1954) described an animal model of hypoxia-induced retinal neovascularization, allowing studies of the disease process, specifically the role of oxygen in vessel loss and the role of hypoxia in vessel growth. Based on this work, other animal models followed, eventually giving rise to genetically manipulated models to study angiogenesis. Ashton's research article titled “Retinal Neovascularization in Health and Disease” is one of the first papers to postulate that hypoxia triggers the production of soluble, secreted angiogenic factors (Am. J. Ophthlamol. 44: 7-24, 1957). Later, Shweiki and colleagues show that hypoxia induces VEGF release in the eye, leading to angiogenesis (Nature 359: 843-845, 1992). They also show that VEGF probably functions as a hypoxia-inducible angiogenic factor, in that VEGF mRNA levels are dramatically increased within a few hours of exposing different cell cultures to hypoxia and return to background when normal oxygen supply is resumed. These findings have great clinical relevance and have led directly to current attempts at treating vascular eye disease with VEGF antagonists.

It is an object of the present invention to modulate (e.g. inhibit) angiogenesis in a number of pathological conditions, especially those non-neoplastic pathological conditions.

Cardiac glycosides are found in a diverse group of plants including Digitalis purpurea and Digitalis lanata (foxgloves), Nerium oleander (common oleander), Thevetia peruviana (yellow oleander), Convallaria majalis (lily of the valley), Urginea maritima and Urginea indica (squill), and Strophanthus gratus (ouabain). Ancient Egyptians and Romans first used plants containing cardiac glycosides medicinally as emetics and for heart ailments. Toxicity from herbal cardiac glycosides was well recognized by 1785, when William Withering published his classic work describing therapeutic uses and toxicity of foxglove, D purpurea. Since the ancient times, therapeutic use of herbal cardiac glycosides continues to be a source of toxicity today. Recently, D lanata mistakenly was substituted for plantain in herbal products marketed to cleanse the bowel; human toxicity resulted. Cardiac glycosides also have been found in Asian herbal products and have been a source of human toxicity. Significant toxicity usually is resultant of depression and a suicide attempt. Toxicity may occur after consuming teas brewed from plant parts or after consuming leaves, flowers, or seeds from plants containing cardiac glycosides. Excessive amounts of cardiac glycosides has been associated with hypertension.

Modem understanding of digitalis therapy arose 50 years ago, when in 1953 Schatzmann (Helv. Physiol. Pharmacol. Acta 11: 346-354, 1953) discovered that cardiotonic steroids are specific inhibitors of the sodium pump and that the digitalis receptor is the Na⁺/K⁺-ATPase of plasma membranes (Skou, Biochim. Biophys. Acta 23: 394-401, 1957). The discovery of the Na⁺/Ca2⁺ exchanger in the late 1960s in mammalian cardiac muscle led to the view that the inhibition of the sodium pump by cardiotonic steroids leads to an increase in the concentration of intracellular Ca²⁺ as a secondary event, which in turn results in a positive inotropic effect on cardiac muscle. This model has been recently refined: it is clear now that the 1 isoform of the sodium pump is ubiquitously distributed in plasma membranes of cardiomyocytes but that the 2/3 isoforms reside in plasma membrane areas close to the endoplasmic reticulum. Such “plasmerosomes” also contain the Na⁺/Ca²⁺ exchanger protein. Inhibition of the 2 and 3 isoforms of Na⁺/K⁺-ATPase in such a restricted area leads to a change in cytosolic Na⁺ and, indirectly, Ca²⁺ concentrations. This modulates in turn the Ca²⁺ content of the sarcoplasmic reticulum and Ca²⁺ signaling, and leads finally to the positive inotropic effect of cardiac glycosides (Blaustein et al., Clin. Exp. Hypertens. 20: 691-703, 1998; Juhaszova & Blaustein, Proc. Natl Acad. Sci. USA 94: 1800-1805, 1997) and an altered gene expression of proteins.

The search for an endogenous digitalis-like compound was aided in the last few decades when it became apparent that volume-expanded forms of hypertension may lead to the release of a natriuretic hormone. The Dahl-deWardener-Blaustein concept (Dahl et al., J. Exp. Med. 126: 687-699, 1967; deWardener & Clarkson, Physiol. Rev. 65: 658-759, 1985; Blaustein, Am. J. Physiol. 232: C167-C173, 1977) of a natriuretic hormone proposes that an enhanced production of endogenous inhibitor(s) of the sodium pump occurs with the adaptive function of decreasing the volume of circulating fluid by means of inhibition of the Na⁺/K⁺-ATPase in renal tubules. The increased production of endogenous digitalis-like compounds would also contribute to hypertension by means of inhibition of Na⁺/K⁺-ATPase in cardiovascular tissues (supra). Hamlyn et al. were the first to demonstrate that the concentration of a circulating factor in blood plasma inhibiting purified Na⁺/K⁺-ATPase correlated with the blood pressure of the donors (Hamlyn et al., Nature 300: 650-652, 1982). This observation paved the way for the identification of endogenous “digitalis” as a group of cardenolides and bufadienolides with related physiological and pathophysiological functions. Today, ouabain, long-known inhibitor of the sodium pump, as well as its related cardiac glycosides have been identified in blood plasma, adrenal glands, and the hypothalamus of mammals. These compounds regulate the activity of Na⁺/K⁺-ATPase-associated cardiac glycoside signaling pathway.

It is another object of the present invention to provide agents and methods for the treatment of depression, hypertension, and all symptoms associated with excessive levels of cardiac glycosides, such as digitalis poisoning, using antagonists of cardiac glycosides.

### SUMMARY OF THE INVENTION

The instant invention is partly based on the discovery that organisms' response to hypoxic stress is regulated by the cardiac-glycoside-steroid signaling pathway. At the macro level, the cardiac-glycoside-steroid signaling pathway controls physiological responses such as reduction of heart rate and hypertension (i.e., increase in blood pressure), in a manner to ensure survival of major organs (e.g., remove or redirect blood flow away from the body extremities, and thus preserving major organs and their functions). At the micro level, the pathway prevents the normal hypoxic response in which cells undergo to recruit blood vessels (e.g. inhibition of VEGF secretion and/or angiogenesis), therefore separating systematic hypoxic response from local hypoxic response. For example, it was found that under hypoxic conditions, mice produce endogenous cardiac glycosides in hypothalamus and adrenal glands. In addition, cardiac glycosides are found to be produced in completely avascular organs, such as human lens.

The invention is also partly based on the discovery that cardiac glycosides do have anti-VEGF and anti HIF activity at the molecular level. Dysregulation (e.g. excessive or insufficient signaling) of the cardiac-glycoside-steroid signaling pathway could be the cause of major disorders such as hypertension, depression, all the symptoms associated with digitalis poising, as well as all symptoms associated with irregular angiogenesis. Thus modulators (e.g. agonists and antagonists) of the cardiac-glycoside-steroid signaling pathway may be used to treat, alleviate, prevent, or control the progress of these conditions/disorders.

Thus in one aspect, the invention provides a host of Na⁺/K⁺-ATPase inhibitors or cardiac glycoside agonists that inhibits the signaling of the cardiac-glycoside-steroid signaling pathway, and methods of using such agonists in treating a number of conditions or disorders. As used herein, “agonist” means a Na⁺/K⁺-ATPase inhibitor (e.g. a ouabain-like cardiac glycoside) that binds to at least one isoform of a Na⁺/K⁺-ATPase, and substantially inhibits the activity of the Na⁺/K⁺-ATPase and/or down-regulates the cardiac-glycoside-steroid signaling pathway and/or hypoxic response in vivo. The in vivo effects of such agonists may include one or more of: increased sympathetic activity, increased blood pressure, and increased heart rate.

In certain embodiments, the agonist's ability to bind and inhibit at least one isoform of a Na⁺/K⁺-ATPase is determined by at least one biological assays. Suitable assays may include: (a) displacement of the specific ³H-ouabain binding from the Na⁺/K⁺-ATPase receptor purified according to Jorghensen (Jorghensen, BBA 356: 36, 1974) and Erdmann (Erdmann et al., Arzneim. Forsh. 34 (II), 1314, 1984) (Recombinant enzymes may also be used); or (b) direct binding of the candidate agonist to the Na⁺/K⁺-ATPase using any art-recognized means (such as BIACORE); or (c) inhibition of the activity of the purified or recombinant Na⁺/K⁺-ATPase measured as % of hydrolysis of ³²P-ATP in presence and in absence of the tested compound (Mall et al., Biochem. Pharmacol. 33: 47, 1984). All references incorporated herein by reference. In certain embodiments, the agonists inhibits at least about 30%, 40%, 50%, 60%, 70%, 80%, 90%, 95%, 99% or more of the Na⁺/K⁺-ATPase activity.

In certain embodiments, the agonist's ability to inhibit or down-regulate hypoxic response in vivo is measured by at least one bioassays, such as the ability of a candidate agonist to increase systolic blood pressure (SBP) and heart rate (HR), such as measured by an indirect tail-cuff method in rats before (basal values)/after treatment. See EP0576915A2 and EP0583578A2 (entire contents incorporated herein by reference). In certain embodiments, the agonists increases HR and/or SBP by at least about 30%, 40%, 50%, 60%, 70%, 80%, 90%, 95%, 99% or more compared to control.

In a related aspect, the invention provides a host of cardiac glycoside antagonists, and methods of using such antagonists in treating a number of conditions or disorders. As used herein, “antagonist,” or “antagonist of cardiac glycoside/Na⁺/K⁺-ATPase inhibitor,” or “cardiac glycoside/Na⁺/K⁺-ATPase inhibitor antagonist” (or their grammatical variations) means an antagonist of a Na⁺/K⁺-ATPase inhibitor (e.g. antagonist of a cardiac glycoside), which inhibitor inhibits the activity of at least one isoform of a Na⁺/K⁺-ATPase, and which antagonist substantially up-regulates hypoxic response in vivo. The antagonists may, without limitation, directly bind to either the Na⁺/K⁺-ATPase, or a ligand or agonist of the Na⁺/K⁺-ATPase, or both, and interferes with the productive interaction of the Na⁺/K⁺-ATPase and its ligand or agonist, thus preventing/inhibiting the agonist/Na⁺/K⁺-ATPase-mediated steroid cardiac glycoside signaling pathway. In other words, the antagonists counter the function of agonists.

In certain embodiments, the antagonist's ability to inhibit/interfere with the binding of an agonist to at least one isoform of a Na⁺/K⁺-ATPase is determined using at least one biological assays. Suitable assays may include: (a) ability of the antagonist to displace specific ³H-ouabain binding from the Na⁺/K⁺-ATPase receptor purified based on Jorghensen (Jorghensen, BBA 356: 36, 1974) and Erdmann (Erdmann et al., Arzneim. Forsh. 34 (11): 1314, 1984) (recombinant enzyme can also be used); (b) ability of the antagonist to prevent an unlabeled agonist to displace specific ³H-ouabain binding from the purified Na⁺/K⁺-ATPase receptor; (c) direct binding of the antagonist to an agonist and/or a Na⁺/K⁺-ATPase, using any art-recognized methods; or (d) inhibition of the activity of ouabain (an agonist) on the purified Na⁺/K⁺-ATPase measured as % of hydrolysis of ³²P-ATP in presence and in absence of the candidate antagonist (Mall et al., Biochem. Pharmacol. 33: 47, 1984). All references incorporated herein by reference.

In certain embodiments, the antagonist's ability to up-regulates hypoxic response in vivo is measured by at least one bioassays, such as the ability of a candidate antagonist to lower blood pressure in adult hypertensive MHS rats, as measured by systolic blood pressure (SBP) and heart rate (HR), such as measured by an indirect tail-cuff method in three-month old hypertensive MHS rats before beginning treatment (basal values). See EP0576915A2 and EP0583578A2 (entire contents incorporated herein by reference). In certain embodiments, the antagonist lowers SBP and/or HR at least about 30%, 40%, 50%, 60%, 70%, 80%, 90%, 95%, 99% or more in at least one of the bioassays.

Thus in one aspect, a salient feature of the present invention is the discovery of a method of inhibiting angiogenesis, such as hypoxia-induced angiogenesis, comprising administering an effective amount of a Na⁺/K⁺-ATPase inhibitor, such as a cardiac glycoside agonist (e.g. ouabain or proscillaridin, etc.), so as to reduce the apparent biological activity (e.g. expression and/or secretion) of VEGF, and other factors having angiogenesis-stimulating activity.

The invention further provides a method of treating an angiogenic disease, comprising administering an effective amount of a Na⁺/K⁺-ATPase inhibitor, such as a cardiac glycoside agonist (e.g. ouabain or proscillaridin, etc.), so as to reduce expression and/or secretion of VEGF, and other factors having angiogenesis-stimulating activity.

The present methods can be used to inhibit angiogenesis which is nonpathogenic; i.e., angiogenesis which results from normal biological processes in the subject. Besides during embryogenesis, angiogenesis is also activated in the female reproductive system during the development of follicles, corpus luteum formation and embryo implantation. During these processes, angiogenesis is mediated mainly by VEGF. Uncontrolled angiogenesis may underlie various female reproductive disorders, such as prolonged menstrual bleeding or infertility, and excessive endothelial cell proliferation has been observed in the endometrium of women with endometriosis. Neovascularization also plays a critical role in successful wound healing that is probably regulated by IL-8 and the growth factors FGF-2 and VEGF. Macrophages, known cellular components of the accompanying inflammatory response, may contribute to the healing process by releasing these angiogenic factors. Examples of non-pathogenic angiogenesis include endometrial neovascularization, and processes involved in the production of fatty tissues or cholesterol. Thus, the invention provides a method for inhibiting non-pathogenic angiogenesis, e.g., for controlling weight or promoting fat loss, for reducing cholesterol levels, or as an abortifacient.

The present methods can also inhibit angiogenesis which is associated with an angiogenic disease; i.e., a disease in which pathogenicity is associated with inappropriate or uncontrolled angiogenesis. For example, most cancerous solid tumors generate an adequate blood supply for themselves by inducing angiogenesis in and around the tumor site. This tumor-induced angiogenesis is often required for tumor growth, and also allows metastatic cells to enter the bloodstream.

Other angiogenic diseases include retinal neovascularization, diabetic retinopathy, retinopathy of prematurity (ROP), endometriosis, macular degeneration, age-related macular degeneration (ARMD), psoriasis, arthritis, rheumatoid arthritis (RA), atherosclerosis, hemangioma, Kaposi's sarcoma, thyroid hyperplasia, Grave's disease, arterioyenous malformations (AVM), vascular restenosis, dermatitis, hemophilic joints, hypertrophic scars, synovitis, vascular adhesions, and other inflammatory diseases. Most, if not all of these diseases are characterized by the destruction of normal tissue by newly formed blood vessels in the area of (diseased) neovascularization. For example, in ARMD, the choroid is invaded and destroyed by capillaries. The angiogenesis-driven destruction of the choroid in ARMD eventually leads to partial or full blindness.

A further aspect of the invention provides a method for inhibiting angiogenesis in lung cancer tissue of a patient, the method comprising administering to the tissue of the patient a composition containing a Na⁺/K⁺-ATPase inhibitor, such as a cardiac glycoside (e.g. ouabain or proscillaridin, etc.), at an amount/level sufficient to down-regulate VEGF expression so as to inhibit angiogenesis in the tissue.

Yet another aspect of the invention provides a method to treat RA in a patient. The method involves administering to synovial tissue of a bone joint of the patient a composition containing a Na⁺/K⁺-ATPase inhibitor, such as a cardiac glycoside (e.g. ouabain or proscillaridin, etc.), at an amount/level sufficient to down-regulate VEGF expression in synovial tissue and inhibit angiogenesis in the synovial tissue.

Yet another aspect of the invention provides a method to treat diabetic retinopathy in a patient. This method involves administering to a retina of the patient a composition containing a Na⁺/K⁺-ATPase inhibitor, such as a cardiac glycoside (e.g. ouabain or proscillaridin, etc.), at an amount/level sufficient to down-regulate VEGF expression in the retina and inhibit angiogenesis in the retina.

Yet another aspect of the invention provides a method to treat choroidal neovascularization in a patient. This method involves delivering to subretinal space or retinal pigment epithelium of the patient a composition containing a Na⁺/K⁺-ATPase inhibitor, such as a cardiac glycoside (e.g. ouabain or proscillaridin, etc.), at an amount/level sufficient to down-regulate VEGF expression in said tissue and inhibit angiogenesis in the choroidal tissue.

A salient feature of the present invention is the discovery that certain agents induce an hypoxic stress response and expression of angiogenic factors (such as VEGF) in cells, and that Na⁺/K⁺-ATPase inhibitors, such as cardiac glycoside agonists, can be used to reduce that response. Since hypoxic stress response is associated with the expression of certain angiogenesis factors, including (but not limited to) VEGF, inhibiting hypoxic stress response would also inhibit VEGF- (and other angiogenesis factor-) mediated angiogenesis.

One aspect of the invention provides a pharmaceutical formulation comprising a Na⁺/K⁺-ATPase inhibitor, such as a cardiac glycoside, and an anti-cancer agent that induces an hypoxic stress response in tumor cells, formulated in a pharmaceutically acceptable excipient and suitable for use in humans to treat a neoplastic disorder.

Another aspect of the invention provides a pharmaceutical formulation comprising a Na⁺/K⁺-ATPase inhibitor, such as a cardiac glycoside, with or without other agents that inhibits hypoxic stress response and/or angiogenic factor (e.g. VEGF) expression in cells, formulated in a pharmaceutically acceptable excipient and suitable for use in humans to treat a non-neoplastic disorder, such as diabetic retinopathy, age-related macular degeneration (ARMD), psoriasis, rheumatoid arthritis (RA) and other inflammatory diseases.

Another aspect of the invention provides a pharmaceutical formulation comprising a Na⁺/K⁺-ATPase inhibitor, either alone or in combination with an anti-angiogenesis agent, formulated in a pharmaceutically acceptable excipient and suitable for use in human patients to reduce angiogenesis.

Another aspect of the invention provides a kit for treating a patient having a neoplastic disorder, comprising a Na⁺/K⁺-ATPase inhibitor and an anti-cancer agent that induces an hypoxic stress response in tumor cells, each of which formulated in premeasured doses for conjoint administration to a patient.

Another aspect of the invention provides a kit for treating a patient having a non-neoplastic angiogenic disorder (such as diabetic retinopathy, age-related macular degeneration (ARMD), psoriasis, rheumatoid arthritis (RA) and other inflammatory diseases, etc.), comprising a Na⁺/K⁺-ATPase inhibitor, with or without one or more agents that inhibits angiogenesis in target cells/tissues, each of which formulated in premeasured doses for conjoint administration to a patient.

Another aspect of the invention provides a kit for treating a patient having excessive or undesirable angiogenesis, comprising a Na⁺/K⁺-ATPase inhibitor, either alone or in combination with an anti-angiogenesis agent, (each of which) formulated in premeasured doses for (conjoint) administration to said patient.

Another aspect of the invention provides a method for treating a patient having excessive or undesirable angiogenesis, comprising administering to the patient an effective amount of a Na⁺/K⁺-ATPase inhibitor, either alone or in combination with an anti-angiogenesis agent.

Another aspect of the invention provides a method for promoting treatment of patients having excessive or undesirable angiogenesis, comprising packaging, labeling and/or marketing a Na⁺/K⁺-ATPase inhibitor, either alone or in combination with an anti-angiogenesis agent, for use in (conjoint) therapy for treating said patients.

Another aspect of the invention provides a method for promoting treatment of patients having excessive or undesirable angiogenesis, comprising packaging, labeling and/or marketing an anti-angiogenesis agent to be used in conjoint therapy with a Na⁺/K⁺-ATPase inhibitor for treating the patients.

Still another aspect of the invention provides a method for promoting treatment of patients having a non-neoplastic angiogenesis disorder (such as diabetic retinopathy, age-related macular degeneration (ARMD), psoriasis, rheumatoid arthritis (RA) and other inflammatory diseases, etc.), comprising packaging, labeling and/or marketing a Na⁺/K⁺-ATPase inhibitor to be used in therapy or conjoint therapy for treating a patient having a non-neoplastic angiogenesis disorder, with or without another anti-angiogenesis agent that inhibits angiogenesis in target tissues/cells.

Another aspect of the invention relates to a method for promoting treatment of patients having a non-neoplastic angiogenesis disorder, comprising packaging, labeling and/or marketing an anti-angiogenesis agent to be used in conjoint therapy with a Na⁺/K⁺-ATPase inhibitor for treating a patient having a non-neoplastic angiogenesis disorder.

In certain embodiments, the angiogenesis is induced by hypoxia., or occurs in a non-pathogenic or non-neoplastic condition.

In certain preferred embodiments, the Na⁺/K⁺-ATPase inhibitor is a cardiac glycoside.

In certain embodiments, the cardiac glycoside, in combination with the anti-angiogenesis agent, has an IC₅₀ for inhibiting angiogenesis or one or more different endothelial cell lines that is at least 2 fold less relative to the corresponding IC₅₀ of the cardiac glycoside alone, and even more preferably at least 5, 10, 50 or even 100 fold less.

In certain embodiments, the cardiac glycoside, in combination with the anti-angiogenesis agent, has an EC₅₀ for inhibiting angiogenesis or one or more different endothelial cell lines that is at least 2 fold less relative to the EC₅₀ of the cardiac glycoside alone, and even more preferably at least 5, 10, 50 or even 100 fold less.

In certain embodiments, the cardiac glycoside has an IC₅₀ for inhibiting migration, proliferation or capillary-forming function of one or more different endothelial cell lines of 500 nM or less, and even more preferably 200 nM, 100 nM, 10 nM or even 1 nM or less. The inhibition of endothelial cell proliferation and/or function may be defined as 20%, 30%, 40%, 50%, 60%, 70%, or 80% of wild-type level.

In certain embodiments, the cardiac glycoside comprises a steroid core with either a pyrone substituent at C17 (the “bufadienolides form”) or a butyrolactone substituent at C17 (the “cardenolide” form).

In certain embodiments, the cardiac glycoside is represented by the general formula:

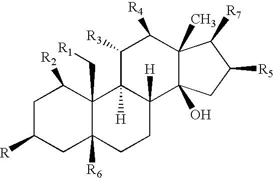

wherein

R represents a glycoside of 1 to 6 sugar residues;

R₁ represents hydrogen, —OH or ═O;

R₂, R₃, R₄, R₅, and R₆ each independently represents hydrogen or —OH;

R₇ represents

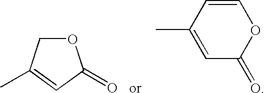

which cardiac glycoside agonist has an IC₅₀ for inhibiting proliferation or function of one or more different endothelial cell lines of 500 nM or less.

In certain preferred embodiments, the sugar residues are selected from L-rhamnose, D-glucose, D-digitoxose, D-digitalose, D-digginose, D-sarmentose, L-vallarose, and D-fructose. In certain embodiments, these sugars are in the β-conformation. The sugar residues may be acetylated, e.g., to effect the lipophilic character and the kinetics of the entire glycoside. In certain preferred embodiments, the glycoside is 1-4 sugar residues in length.

In certain embodiments, the cardiac glycoside is selected from digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocymarin, digoxin (CP 4072), strophanthidin oxime, strophanthidin semicarbazone, strophanthidinic acid lactone acetate, ernicyrnarin, sannentoside D, sarverogenin, sarmentoside A, sarmentogenin, or a pharmaceutically acceptable salt, ester, amide, or prodrug thereof In certain preferred embodiments, the cardiac glycoside is ouabain or proscillaridin.

In certain embodiments, the Na⁺/K⁺-ATPase inhibitor inhibits the expression of an angiogenesis factor in said patient.

In certain embodiments, the expression of the angiogenesis factor is induced or up-regulated by hypoxia.

In certain embodiments, the expression of the angiogenesis factor is induced or up-regulated by HIF-1α.

In certain embodiments, the the angiogenesis factor is VEGF.

In certain embodiments, the non-neoplastic condition is: retinal neovascularization, diabetic retinopathy, retinopathy of prematurity (ROP), endometriosis, macular degeneration, age-related macular degeneration (ARMD), psoriasis, arthritis, rheumatoid arthritis (RA), atherosclerosis, hemangioma, Kaposi's sarcoma, thyroid hyperplasia, Grave's disease, arterioyenous malformations (AVM), vascular restenosis, dermatitis, hemophilic joints, hypertrophic scars, synovitis, Alzheimer's Disease, obesity, diabete, vascular adhesions, or other inflammatory diseases.

Another aspect of the invention provides a method of inhibiting angiogenesis, such as hypoxia-induced angiogenesis, comprising administering an effective amount of a Na⁺/K⁺-ATPase inhibitor, such as a cardiac glycoside agonist (e.g. ouabain or proscillaridin, etc.), so as to reduce the apparent biological activity (e.g. expression and/or secretion) of VEGF, and other factors having angiogenesis-stimulating activity.

Another aspect of the invention provides a method of treating an angiogenic disease/condition, comprising administering to a patient with said disease/condition an effective amount of a Na⁺/K⁺-ATPase inhibitor to reduce expression and/or secretion of VEGF, and other factors having angiogenesis-stimulating activity.

In certain embodiments, the non-pathogenic condition is: endometrial neovascularization, endometriosis, female reproductive disorder associated with excessive angiogenesis (such as prolonged menstrual bleeding or infertility), or a process involved in the production of fatty tissues or cholesterol.

Other Na⁺/K⁺-ATPase inhibitors are available in the literature. See, for example, U.S. Pat. No. 5,240,714 which describes a non-digoxin-like Na⁺/K⁺-ATPase inhibitory factor. Recent evidence suggests the existence of several endogenous Na⁺/K⁺-ATPase inhibitors in mammals and animals. For instance, marinobufagenin (3,5-dihydroxy-14,15-epoxy bufodienolide) may be useful in the current combinatorial therapies.

Those skilled in the art can also rely on screening assays to identify compounds that have Na⁺/K⁺-ATPase inhibitory activity. PCT Publications WO00/44931 and WO02/42842, for example, teach high-throughput screening assays for modulators of Na⁺/K⁺-ATPases.

The Na⁺/K⁺-ATPase consists of at least two dissimilar subunits, the large α subunit with all known catalytic functions and the smaller glycosylated β subunit with chaperonic function. In addition there may be a small regulatory, so-called FXYD-peptide. Four α peptide isoforms are known and isoform-specific differences in ATP, Na⁺ and K⁺ affinities and in Ca²⁺ sensitivity have been described. Thus changes in The Na⁺/K⁺-ATPase isoform distribution in different tissues, as a function of age and development, electrolytes, hormonal conditions etc. may have important physiological implications. Cardiac glycosides like ouabain are specific inhibitors of the Na⁺/K⁺-ATPase. The four α peptide isoforms have similar high ouabain affinities with K_{d} of around 1 nM or less in almost all mammalian species. In certain embodiments, the Na⁺/K⁺-ATPase inhibitor is more selective for complexes expressed in non-cardiac tissue, relative to cardiac tissue.

In certain embodiments, the subject Na⁺/K⁺-ATPase inhibitors (e.g. cardiac glycosides) can also be combined with a therapeutically effective amount of another molecule which negatively regulates angiogenesis which may be, but is not limited to, VEGF inhibitors such as antibodies against VEGF or antigenic epitopes thereof; soluble VEGF receptors such as Flt-1, Flk-1/KDR, Flt-4, neuropilin-1 and -2 (NP1 and NP2); TNP-470; PTK787/ZK 222584 (1-[4chloroanilino]-4-[4-pyridylmethyl]ph-thalazine succinate) (Novartis International AG, Basel, Switzerland); VEGF receptor inhibitors, such as SU5416, or antibodies against such receptors such as DC101 (ImClone Systems, Inc., NY); tyrosine kinase inhibitors; prolactin (16-KDs fragment), angiostatin (38-kD fragment of plasminogen), endostatin, basic fibroblast derived growth factor (bFGF) inhibitors such as a soluble bFGF receptor; transforming growth factor beta; interferon alfa; epidermal-derived growth factor inhibitors; platelet derived growth factor inhibitors; an integrin blocker, interleukin-12; troponin-1; 12-lipoxygenase (LOX) inhibitors, such as BHPP (N-benzyl-N-hydroxy-5-phenylpentanamide) (Nie et al. Blood 95:2304-2311); platelet factor 4; thrombospondin-1; tissue inhibitors of metalloproteases such as TIMP1 and TIMP2; transforming growth factor beta; interferon alfa; protamine; combination of heparin and steroids; and steroids such as tetrahydrocortisol; which lack gluco- and mineral-corticoid activity; angiostatin; phosphonic acid agents; anti-invasive factor; retinoic acids and derivatives thereof; paclitaxel (U.S. Pat. No. 5,994,341); interferon-inducible protein 10 and fragments and analogs of interferon-inducible protein 10; medroxyprogesterone; sulfated protamine; prednisolone acetate; herbimycin A; peptide from retinal pigment epithelial cell; sulfated polysaccharide; and phenol derivatives; isolated body wall of a sea cucumber, the isolated epithelial layer of the body-wall of the sea cucumber, the flower of the sea cucumber, their active derivatives or mixtures thereof; thalidomide and various related compounds such as thalidomide precursors, analogs, metabolites and hydrolysis products; 4 kDa glycoprotein from bovine vitreous humor; a cartilage derived factor, human interferon-alpha; ascorbic acid ethers and related compounds; sulfated polysaccharide DS 4152; and a synthetic fumagillin derivative, AGM 1470. In the preferred embodiment of the present invention, the angiogenesis inhibitor is a VEGF inhibitor. Most preferably the angiogenesis inhibitor is truncated, soluble form of a VEGF receptor.

In other embodiments, the angiogenesis inhibitors can be: Angioarrestin; Angiostatin (plasminogen fragment); Antiangiogenic antithrombin III; Cartilage-derived inhibitor (CDI); CD59 complement fragment; Endostatin (collagen XVIII fragment); Fibronectin fragment; Gro-beta; Heparinases; Heparin hexasaccharide fragment; Human chorionic gonadotropin (hCG); Interferon alpha/beta/gamma; Interferon inducible protein (IP-10); Interleukin-4; Interleukin-12; Kringle 5 (plasminogen fragment); Metalloproteinase inhibitors (TIMPs); 2-Methoxyestradiol; PEDF; Placental ribonuclease inhibitor; Plasminogen activator inhibitor; Platelet factor-4 (PF4); Prolactin 16 kD fragment; Proliferin-related protein (PRP); Retinoids; Tetrahydrocortisol-S; Thrombospondin-1 (TSP-1); Troponin I; Transforming growth factor-beta (TGF-b); VEGI; Vasculostatin; Vasostatin (calreticulin fragment), or combinations thereof.

In other embodiments, the angiogenesis inhibitors can be an inhibitor (antibody, antisense of the gene, RNAI agent against the gene, small molecule inhibitor, dominant negative binder, small peptide fragment or peptidiomimetics, etc.) of any one or more of the following angiogenesis stimulators: Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; Transforming growth factor-alpha (TGF-alpha); Transforming growth factor-beta (TGF-beta); Tumor necrosis factor-alpha (TNF-alpha); or Vascular endothelial growth factor (VEGF)/vascular permeability factor (VPF).

In certain embodiments, the anti-angiogenesis agent is: a VEGF inhibitor; a soluble VEGF receptor; TNP-470; PTK787/ZK 222584 (1-[4chloroanilino]-4-[4-pyridylmethyl]ph-thalazine succinate); a VEGF receptor inhibitor (such as SU5416, or antibodies against such receptors such as DC101); a tyrosine kinase inhibitor; prolactin (16-KDs fragment); angiostatin (38-kD fragment of plasminogen); endostatin; a basic fibroblast derived growth factor (bFGF) inhibitor (such as a soluble bFGF receptor); transforming growth factor beta; interferon alpha; an epidermal-derived growth factor inhibitor; a platelet derived growth factor (PDGF) inhibitor; an integrin blocker; interleukin-12; troponin-1; a 12-lipoxygenase (LOX) inhibitor (such as BHPP (N-benzyl-N-hydroxy-5-phenylpentanamide)); platelet factor 4; thrombospondin-1; a tissue inhibitor of metalloproteases (such as TIMP1 and TIMP2); protamine; a combination of heparin and steroids; a steroid lacking gluco- and mineral-corticoid activity (such as tetrahydrocortisol); a phosphonic acid agent; an anti-invasive factor; a retinoic acid and derivatives thereof; paclitaxel; interferon-inducible protein 10 and fragments and analogs thereof; medroxyprogesterone; sulfated protamine; prednisolone acetate; herbimycin A; a peptide from retinal pigment epithelial cell; a sulfated polysaccharide; a phenol derivative; isolated body wall, isolated epithelial layer of the body-wall, or flower of a sea cucumber, or their active derivatives or mixtures thereof; thalidomide and thalidomide precursors, analogs, metabolites and hydrolysis products; a 4 kDa glycoprotein from bovine vitreous humor; a cartilage derived factor; an ascorbic acid ether and related compounds; sulfated polysaccharide DS 4152; and a synthetic fumagillin derivative, AGM 1470.

In certain embodiments, the The pharmaceutical formulation of claim 1, kit of claim 2, or method of claim 3, 4, or 5, wherein the patient has, or is at risk for developing abnormal proliferation of fibrovascular tissue, acne rosacea, acquired immune deficiency syndrome, artery occlusion, atopic keratitis, bacterial ulcers, Bechets disease, blood borne tumors, carotid obstructive disease, chemical burns, choroidal neovascularization, chronic inflammation, chronic retinal detachment, chronic uveitis, chronic vitritis, contact lens overwear, corneal graft rejection, corneal neovascularization, corneal graft neovascularization, Crohn's disease, Eales disease, epidemic keratocon junctivitis, fungal ulcers, Herpes simplex infections, Herpes zoster infections, hyperviscosity syndromes, Kaposi's sarcoma, leukemia, lipid degeneration, Lyme's disease, marginal keratolysis, Mooren ulcer, Mycobacteria infections other than leprosy, myopia, ocular neovascular disease, optic pits, Osler-Weber syndrome (Osler-Weber-Rendu, osteoarthritis, Pagets disease, pars planitis, pemphigoid, phylectenulosis, polyarteritis, post-laser complications, protozoan infections, pseudoxanthoma elasticum, pterygium keratitis sicca, radial keratotomy, retinal neovascularization, retinopathy of prematurity, retrolental fibroplasias, sarcoid, scleritis, sickle cell anemia, Sogrens syndrome, solid tumors, Stargarts disease, Steven's Johnson disease, superior limbic keratitis, syphilis, systemic lupus, Terrien's marginal degeneration, toxoplasmosis, trauma, tumors of Ewing sarcoma, tumors of neuroblastoma, tumors of osteosarcoma, tumors of retinoblastoma, tumors of rhabdomyosarcoma, ulceritive colitis, vein occlusion, Vitamin A deficiency or Wegeners sarcoidosis.

In certain embodiments, the patient has, or is at risk for developing diabetes; parasitic disease; abnormal wound healing; hypertrophy following surgery, burns, injury or trauma; inhibition of hair growth; inhibition of ovulation and corpus luteum formation; inhibition of implantation or inhibition of embryo development in the uterus.

In certain embodiments, the patient has, or is at risk for developing graft rejection, lung inflammation, nephrotic syndrome, preeclampsia, edema associated with brain tumors, ascites associated with malignancies, Meigs' syndrome, pericardial effusion, pericarditis or pleural effusion.

In certain embodiments, the anti-angiogenesis agent is: Angioarrestin; Angiostatin (plasminogen fragment); Antiangiogenic antithrombin III; Cartilage-derived inhibitor (CDI); CD59 complement fragment; Endostatin (collagen XVIII fragment); Fibronectin fragment; Gro-beta; Heparinases; Heparin hexasaccharide fragment; Human chorionic gonadotropin (hCG); Interferon alpha/beta/gamma; Interferon inducible protein (IP-10); Interleukin-4; Interleukin-12; Kringle 5 (plasminogen fragment); Metalloproteinase inhibitors (TIMPs); 2-Methoxyestradiol; PEDF; Placental ribonuclease inhibitor; Plasminogen activator inhibitor; Platelet factor-4 (PF4); Prolactin 16 kD fragment; Proliferin-related protein (PRP); Retinoids; Tetrahydrocortisol-S; Thrombospondin-1 (TSP-1); Troponin I; Transforming growth factor-beta (TGF-b); Vasculostatin; VEGI; Vasostatin (calreticulin fragment), or combinations thereof.

In certain embodiments, the anti-angiogenesis agent is an inhibitor (antibody, antisense of the gene, RNAi agent against the gene, small molecule inhibitor, dominant negative binder, small peptide fragment or peptidiomimetics, etc.) of any one or more of angiogenesis stimulators selected from: Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; Transforming growth factor-alpha (TGF-alpha); Transforming growth factor-beta (TGF-beta); Tumor necrosis factor-alpha (TNF-alpha); or Vascular endothelial growth factor (VEGF)/vascular permeability factor (VPF).

In certain embodiments, the inhibitor is in the form of an antibody, an antisense oligonucleotide or vector encoding the antisense oligonucleotide, an RNAi agent (siRNA, short hairpin RNA) or vector encoding the RNAi agent, a small molecule inhibitor (e.g. with molecular weight less than 3000 kDa), a dominant negative binder, or a small peptide fragment or peptidiomimetics thereof.

The compound, pharmaceutical composition, method, and kit of the invention can also be used to treat all other hypoxia-related conditions, including hypoxia-induced dementia such as Alzheimer's Disease.

Another aspect of the invention provides the use of cardiac glycoside antagonists to treat conditions associated with the steroid cardiac glycoside signaling activity at the presence of excessive cardiac glycoside agonists (e.g. ouabain etc.).

Thus in one aspect, the invention provides a method of treating/preventing a condition associated with excessive amount of cardiac glycosides in a patient, comprising administering to the patient an effective amount of a cardiac glycoside inhibitor or antagonist.

In certain embodiments, the condition is hypoxia-induced dementia, such as Alzheimer's disease.

Another aspect of the invention provides a pharmaceutical formulation comprising a Na⁺/K⁺-ATPase inhibitor, either alone or in combination with an agent effective for treating or preventing Alzheimer's Disease (AD), formulated in a pharmaceutically acceptable excipient and suitable for use in human patients to treat or prevent AD.

Another aspect of the invention provides a kit for treating a patient having or in risk of having Alzheimer's Disease, comprising a Na⁺/K⁺-ATPase inhibitor, either alone or in combination with an agent effective for treating or preventing Alzheimer's Disease (AD), (each of which) formulated in premeasured doses for (conjoint) administration to said patient.

Another aspect of the invention provides a method for treating a patient having or in risk of having Alzheimer's Disease, comprising administering to the patient an effective amount of a Na⁺/K⁺-ATPase inhibitor, either alone or in combination with an agent effective for treating or preventing Alzheimer's Disease (AD).

Another aspect of the invention provides a method for promoting treatment of patients having or in risk of having Alzheimer's Disease, comprising packaging, labeling and/or marketing a Na⁺/K⁺-ATPase inhibitor, either alone or in combination with an agent effective for treating or preventing Alzheimer's Disease (AD), for use in (conjoint) therapy for treating said patients.

In certain embodiments, the condition includes all symptoms of digitalis poisoning (e.g. one or more of fatigue, visual symptoms, muscle weakness to vertigo, nausea, anorexia, psychic complaints, abdominal pain, dizziness, abnormal dreams, headache, diarrhea, vomiting, syncope, lethargy, seizures, impaired memory, confusion, disorientation, delusions, depression, and delirium, etc.), hypokalemia, bradydysrhythmias and AV blocks, ventricular tachydysrhythmias, atrial tachycardia with block, non-paroxysmal junctional tachycardia, atrioventricular conduction disturbances, first-degree block, sinus impulse formation disturbances, sinus bradycardia, sinus arrest, sinoatrial block, second-degree atrioventricular block, third-degree atrioventricular block, non-Paroxysmal atrial tachycardia with block, non-paroxysmal junctional tachycardia, atrioventricular dissociation, second-degree atrioventricular block (Type I), premature ventricular contractions, ventricular bigeminy, ventricular tachycardia, bidirectional ventricular tachycardia, ventricular fibrillation.

GI symptoms associated with digitalis toxicity are usually the first symptoms to evolve. These symptoms seem nonspecific, and include nausea, vomiting, abdominal pain, diarrhea, and anorexia. Neurological symptoms often include giddiness, headache, dizziness, fatigue, weakness, numbness (especially of tongue and lips), hallucinations, altered mental status (e.g., disorientation, confusion, drowsiness, lethargy), and seizures. Findings may include an altered level of consciousness, hypotonia, hyporeflexia, dysarthria, ataxia, horizontal nystagmus, and generalized seizures. Visual effects include blurred vision, scotomas, and flashes of light. Abnormal color perceptions of yellow or yellow-green halos (e.g., xanthopsia) may occur. Cardiac symptoms including palpitations, fluttering in chest, chest pressure or shortness-of-breath, lightheadedness, dizziness, faintness, and sensation of irregular heartbeat may be noted.

Thus another aspect of the invention provides a pharmaceutical formulation comprising an antagonist of a Na⁺/K⁺-ATPase inhibitor formulated in a pharmaceutically acceptable excipient and suitable for use in human patients to treat digitalis poinsoning.

Another aspect of the invention provides a kit for treating a patient having digitalis poinsoning, comprising an antagonist of a Na⁺/K⁺-ATPase inhibitor, formulated in premeasured doses for administration to said patient.

Another aspect of the invention provides a method for treating a patient having digitalis poinsoning, comprising administering to the patient an effective amount of an antagonist of a Na⁺/K⁺-ATPase inhibitor.

Another aspect of the invention provides a method for promoting treatment of patients having digitalis poinsoning, comprising packaging, labeling and/or marketing an antagonist of a Na⁺/K⁺-ATPase inhibitor for use in therapy for treating said patients.

In certain embodiments, the Na⁺/K⁺-ATPase inhibitor is a cardiac glycoside.

In certain embodiments, the symptoms of the digitalis poisoning to be treated include one or more of: fatigue, visual symptoms, muscle weakness to vertigo, nausea, anorexia, psychic complaints, abdominal pain, dizziness, abnormal dreams, headache, diarrhea, vomiting, syncope, lethargy, seizures, impaired memory, confusion, disorientation, delusions, depression, and delirium, etc.), hypokalemia, bradydysrhythmias and AV blocks, ventricular tachydysrhythmias, atrial tachycardia with block, non-paroxysmal junctional tachycardia, atrioventricular conduction disturbances, first-degree block, sinus impulse formation disturbances, sinus bradycardia, sinus arrest, sinoatrial block, second-degree atrioventricular block, third-degree atrioventricular block, non-Paroxysmal atrial tachycardia with block, non-paroxysmal junctional tachycardia, atrioventricular dissociation, second-degree atrioventricular block (Type I), premature ventricular contractions, ventricular bigeminy, ventricular tachycardia, bidirectional ventricular tachycardia, ventricular fibrillation.

In certain embodiments, the level of cardiac glycosides in the serum of the patient is more than the therapeutic range of cardiac glycosides, such as 0.5 to 2 ng/ml for digoxin. Preferably, the serum level of cardiac glycosides in the intoxicated patients is about 2-5 ng/ml, or more than about 3 ng/ml. However, in certain patients, the serum cardiac glycosides may be as low as about 1 ng/ml or less, and the patients still manifest symptoms of digitalis toxicity. The antagonists of the cardiac glycosides are contemplated to be effective in these patients too.

In certain embodiments, the cardiac glycoside inhibitor/antagonist is an antibody that can bind and/or neutralize the cardiac glycoside. In a preferred embodiment, the cardiac glycoside inhibitor/antagonist is an Fab fragment of an anti-digoxin antibody (e.g. Digiband), which also cross-reacts with ouabain (Huang et al., Circ. Res. 71: 1059- 1066, 1992; Takahashi et al., Jap. Circ. J. 51: 1199-1207, 1987, all contents incorporated herein by reference).

In certain embodiments, these antibody antagonists are therapeutic agents that can be used to treat/alleviate/prevent the conditions associated with excessive amount of agonists.

In certain other embodiments, these antibody antagonists are diagnostic agents that can be used to detect and/or quantitate the agonists associated with certain conditions.

Another aspect of the invention provides a pharmaceutical formulation comprising an antagonist of a Na⁺/K⁺-ATPase inhibitor, either alone or in combination with an anti-depression agent, formulated in a pharmaceutically acceptable excipient and suitable for use in human patients to reduce depression.

Another aspect of the invention provides a kit for treating a patient having or in risk of having depression, comprising an antagonist of a Na⁺/K⁺-ATPase inhibitor, either alone or in combination with an anti-depression agent, (each of which) formulated in premeasured doses for (conjoint) administration to said patient.

Another aspect of the invention provides a method for treating a patient having or in risk of having depression, comprising administering to the patient an effective amount of an antagonist of a Na⁺/K⁺-ATPase inhibitor, either alone or in combination with an anti-depression agent.

Another aspect of the invention provides a method for promoting treatment of or prevention in patients having or in risk of having depression, comprising packaging, labeling and/or marketing an antagonist of a Na⁺/K⁺-ATPase inhibitor, either alone or in combination with an anti-depression agent, for use in (conjoint) therapy for treating said patients.

Another aspect of the invention provides a method for promoting treatment of or prevention in patients having or in risk of having depression, comprising packaging, labeling and/or marketing an anti-depression agent to be used in conjoint therapy with an antagonist of a Na⁺/K⁺-ATPase inhibitor for treating the patients.

In certain embodiments, the Na⁺/K⁺-ATPase inhibitor is a cardiac glycoside.

In certain embodiments, the depression is caused by digitalis poisoning.

In certain embodiments, the antagonist is an antibody for functional fragment thereof (e.g. the Fab fragment of an anti-digoxin Ab Digiband) specific for said Na⁺/K⁺-ATPase inhibitor (e.g., cardiac glycoside).

In certain embodiments, the antagonist is a 17beta-(3-furyl)-5beta-androstane-3beta,14beta, 17alpha-triol derivative or analog.

In certain embodiments, the antagonist is PST 2238.

In certain embodiments, the anti-depression agent is an SSRI (Selective Serotonin Reuptake Inhibitors) selected from: Celexa (citalopram), Lexapro (escitalopram HBr), Luvox (fluvoxamine), Paxil, Paxil CR (paroxetine), Prozac, Prozac Weekly (fluoxetine), Zoloft (sertraline); a tricyclic antidepressant selected from: amitriptyline, desipramine, or nortriptyline; an MAOI (monoamine oxidase Inhibitor) selected from: Nardil (phenelzine), or Parnate (tranylcypromine); or Cymbalta (Duloxetine); Effexor, Effexor XR (venlafaxine); Remeron (mirtazepine); Serzone (nefazodone); Trazodone; Wellbutrin, Wellbutrin SR, Wellbiutrin XL (bupropion).

In certain embodiments, the cardiac glycoside inhibitor/antagonist is an inhibitor of the ouabain-like compounds, such as PST2238 and its related compounds (Ferrari et al., J. Pharmacol. Exp. Therapeut. 285: 83-94, 1998; Quadri et al., J. Med. Chem. 40: 1561-1564, 1997, all contents incorporated herein by reference). Some of these compounds may be competitive inhibitors of one or more cardiac glycoside agonists, such that the antagonists prevent the productive interaction of an agonist and the Na⁺/K⁺-ATPase.

Since there are different isoforms of Na⁺/K⁺-ATPase, and they may exhibit differential tissue (and even subcellular) distribution, the invention also provides tissue/isoform specific cardiac glycoside agonists and/or antagonists that may be selectively used to achieve specific desired biological effects.

It is contemplated that all embodiments described above can be freely combined with one or more other embodiments, even when the embodiments involved are described under different aspects of the invention.

### BRIEF DESCRIPTION OF THE DRAWINGS

FIG. 1. Schematic diagram of using Sentinel Line promoter-less trap vectors to generate active genetic sites expressing drug selection markers and/or reporters.

FIG. 2. Schematic diagram of creating a Sentinel Line by sequential isolation of cells resistant to positive and negative selection drugs.

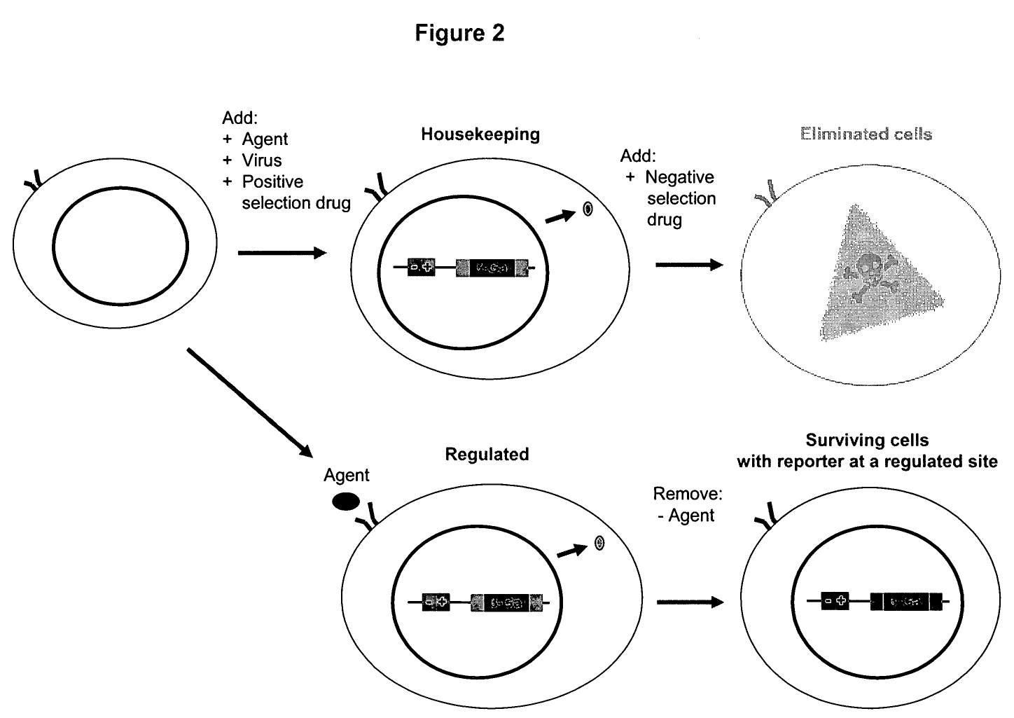

FIG. 3. Adaptation of a cancer cell to hypoxia, which leads to activation of multiple survival factors. The HIF family acts as a master switch transcriptionally activating many genes and enabling factors necessary for glycolytic energy metabolism, angiogenesis, cell survival and proliferation, and erythropoiesis. The level of HIF proteins present in the cell is regulated by the rate of their synthesis in response to factors such as hypoxia, growth factors, androgens and others. Degradation of HIF depends in part on levels of reactive oxygen species (ROS) in the cell. ROS leads to ubiquitylation and degradation of HIF.

FIG. 4. FACS Analysis of Sentinel Lines. Sentinel Lines were developed by transfecting A549 (NSCLC lung cancer) and Panc-1 (pancreatic cancer) cell lines with gene-trap vectors containing E. coli LacZ-encoded β-galactosidase (β-gal) as the reporter gene. The β-gal activity in Sentinel Lines (green) was measured by flow cytometry using a fluorogenic substrate fluoresescein di-beta-D-galactopyranoside (FDG). The autofluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

FIG. 5. Western Blot analysis of HIF1α expression indicates that cardiac glycoside compounds inhibit HIF1α expression.

FIG. 6. Demonstrates that BNC1 inhibits HIF1α synthesis.

FIG. 7. Demonstrates that BNC1 induces ROS production and inhibits HIF-1α induction in tumor cells.

FIG. 8. Demonstrates that the cardiac glycoside compounds BNC1 and BNC4 directly or indirectly inhibits in tumor cells the secretion of the angiogenesis factor VEGF.

FIG. 9. These four charts show FACS analysis of response of a NSCLC Sentinel Line (A549), when treated 40 hrs with four indicated agents. Control (untreated) is shown in purple. Arrow pointing to the right indicates increase in reporter activity whereas inhibitory effect is indicated by arrow pointing to the left. The results indicate that standard chemotherapy drugs turn on survival response in tumor cells.

FIG. 10. Effect of BNC4 on Gemcitabine-induced stress responses visualized by A549 Sentinel Lines™.

FIG. 11. Pharmacokinetic analysis of BNC1 delivered by osmotic pumps. Osmotic pumps (Model 2002, Alzet Inc) containing 200 μl of BNC1 at 50, 30 or 20 mg/ml in 50% DMSO were implanted subcutaneously into nude mice. Mice were sacrificed after 24, 48 or 168 hrs, and plasma was extracted and analyzed for BNC1 by LC-MS. The values shown are average of 3 animals per point.

FIG. 12. Shows effect of BNC1 alone or in combination with standard chemotherapy on growth of xenografted human pancreatic tumors in nude mice.

FIG. 13. Shows anti-tumor activity of BNC1 and Cytoxan against Caki-1 human renal cancer xenograft.

FIG. 14. Shows anti-tumor activity of BNC1 alone or in combination with Carboplatin in A549 human non-small-cell-lung carcinoma.

FIG. 15. Titration of BNC1 to determine minimum effective dose effective against Panc-1 human pancreatic xenograft in nude mice. BNC1 (sc, osmotic pumps) was tested at 10, 5 and 2 mg/ml.

FIG. 16. Combination of BNC1 with Gemcitabine is more effective than either drug alone against Panc-1 xenografts.

FIG. 17. Combination of BNC1 with 5-FU is more effective than either drug alone against Panc-1 xenografts.

FIG. 18. Comparison of BNC1 and BNC4 in inhibiting hypoxia-mediated HIF-1α induction in human tumor cells (Hep3B cells).

FIG. 19. Comparison of BNC1 and BNC4 in inhibiting hypoxia-mediated HIF-1α induction in human tumor cells (Caki-1 and Panc-1 cells).

FIG. 20. BNC4 blocks HIF-1α induction by a prolyl-hydroxylase inhibitor under normoxia.

### DETAILED DESCRIPTION OF THE INVENTION

I. Overview

The present invention is based in part on the discovery that certain agents, such as Na⁺/K⁺-ATPase inhibitors, e.g. cardiac glycoside agonists (ouabain or proscillaridin, etc.) inhibit hypoxia-induced conditions such as angiogenesis, e.g., angiogenesis in the context of hypoxic stress response in tissues/cells, which response includes the up-regulation of expression of certain angiogenesis factors, such as VEGF.

The present invention is also based in part on the ability of certain cardiac glycoside antagonists to block or neutralize the cardiac glycoside signaling in various biological reactions, such as hypertension, depression, etc.

The following sections describe different aspects and embodiments of the invention in more detail.

II. Definitions

As used herein the term “animal” refers to mammals, preferably mammals such as humans. Likewise, a “patient” or “subject” to be treated by the method of the invention can mean either a human or non-human animal.

As used herein, the term “cancer” or “tumor” refers to any neoplastic disorder, including such cellular disorders as, for example, renal cell cancer, Kaposi's sarcoma, chronic leukemia, prostate cancer, breast cancer, sarcoma, pancreatic cancer, ovarian carcinoma, rectal cancer, throat cancer, melanoma, colon cancer, bladder cancer, mastocytoma, lung cancer, mammary adenocarcinoma, myeloma, lymphoma, pharyngeal squamous cell carcinoma, and gastrointestinal or stomach cancer. Preferably, the cancer which is treated in the present invention is melanoma, lung cancer, breast cancer, pancreatic cancer, prostate cancer, colon cancer, or ovarian cancer.

The “growth state” of a cell refers to the rate of proliferation of the cell and the state of differentiation of the cell.

As used herein, “hyperproliferative disease” or “hyperproliferative disorder” refers to any disorder which is caused by or is manifested by unwanted proliferation of cells in a patient. Hyperproliferative disorders include but are not limited to cancer, psoriasis, rheumatoid arthritis, lamellar ichthyosis, epidermolytic hyperkeratosis, restenosis, endometriosis, and abnormal wound healing.

As used herein, “proliferating” and “proliferation” refer to cells undergoing mitosis.

As used herein, “unwanted proliferation” means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyperproliferative disease” or “hyperproliferative disorder.”

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

III. Exemplary Embodiments

Many Na⁺/K⁺-ATPase inhibitors are available in the literature. See, for example, U.S. Pat. No. 5,240,714 which describes a non-digoxin-like Na⁺/K⁺-ATPase inhibitory factor. Recent evidence suggests the existence of several endogenous Na⁺/K⁺-ATPase inhibitors in mammals and animals. For instance, marinobufagenin (3,5-dihydroxy-14,15-epoxy bufodienolide) may be useful in the current combinatorial therapies.

Those skilled in the art can also rely on screening assays to identify compounds that have Na⁺/K⁺-ATPase inhibitory activity. PCT Publications WO00/44931 and WO02/42842, for example, teach high-throughput screening assays for modulators of Na⁺/K⁺-ATPases.

The Na⁺/K⁺-ATPase consists of at least two dissimilar subunits, the large α subunit with all known catalytic functions and the smaller glycosylated β subunit with chaperonic function. In addition there may be a small regulatory, so-called FXYD-peptide. Four α peptide isoforms are known and isoform-specific differences in ATP, Na⁺ and K⁺ affinities and in Ca²⁺ sensitivity have been described. Thus changes in Na⁺/K⁺-ATPase isoform distribution in different tissues, as a function of age and development, electrolytes, hormonal conditions etc. may have important physiological implications. Cardiac glycosides like ouabain are specific inhibitors of the Na⁺/K⁺-ATPase. The four α peptide isoforms have similar high ouabain affinities with K_{d} of around 1 nM or less in almost all mammalian species. In certain embodiments, the Na⁺/K⁺-ATPase inhibitor is more selective for complexes expressed in non-cardiac tissue, relative to cardiac tissue. The following section describes a preferred embodiments of Na⁺/K⁺-ATPase inhibitors—cardiac glycosides.

A. Exemplary Cardiac Glycosides

The inventors have demonstrated that cardiac glycosides are effective in suppressing hypoxia-induced gene expression, such as VEGF expression in cancer cells. For example, cardiac glycosides are effective in suppressing VEGF, EGF, insulin and/or IGF-responsive gene expression in various growth factor responsive cancer cell lines. As another example, the inventors have observed that cardiac glycosides are effective in suppressing HIF-responsive gene expression in cancer cell lines and furthermore, cardiac glycosides are shown to have potent antiangiogenesis effects in certain cell lines.

The term “cardiac glycoside” or “cardiac steroid” is used in the medical field to refer to a category of compounds tending to have positive inotropic effects on the heart. As a general class of compounds, cardiac glycosides comprise a steroid core with either a pyrone or butenolide substituent at C17 (the “pyrone form” and “butenolide form”). Additionally, cardiac glycosides may optionally be glycosylated at C3. Most cardiac glycosides include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a cardiac glycoside with little other effect on biological activity. For this reason, aglycone forms of cardiac glycosides are available and are intended to be encompassed by the term “cardiac glycoside” as used herein. The pharmacokinetics of a cardiac glycoside may be adjusted by adjusting the hydrophobicity of the molecule, with increasing hydrophobicity tending to result in greater absorption and an increased half-life. Sugar moieties may be modified with one or more groups, such as an acetyl group.

A large number of cardiac glycosides are known in the art for the purpose of treating cardiovascular disorders. Given the significant number of cardiac glycosides that have proven to have anticancer effects in the assays disclosed herein, it is expected that most or all of the cardiac glycosides used for the treatment of cardiovascular disorders may also be used for treating proliferative disorders. Examples of preferred cardiac glycosides include ouabain, digitoxigenin, digoxin and lanatoside C. Additional examples of cardiac glycosides include: Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin and calotoxin. Cardiac glycosides may be evaluated for effectiveness in the treatment of cancer by a variety of methods, including, for example: evaluating the effects of a cardiac glycoside on expression of a HIF-responsive gene in a cancer cell line or evaluating the effects of a cardiac glycoside on cancer cell proliferation.

Notably, cardiac glycosides affect proliferation of cancer cell lines at a concentration well below the known toxicity level. The IC₅₀ measured for ouabain across several different cancer cell lines ranged from about 15 nM to about 600 nM, or about 80 nM to about 300 nM. The concentration at which a cardiac glycoside is effective as part of an antiproliferative treatment may be further decreased by combination with an additional agent that negatively regulates HIF-responsive genes, such as a redox effector or a steroid signal modulator. For example, as shown herein, the concentration at which a cardiac glycoside (e.g. ouabain or proscillaridin) is effective for inhibiting proliferation of cancer cells is decreased 5-fold by combination with a steroid signal modulator (Casodex). Therefore, in certain embodiments, the invention provides combination therapies of cardiac glycosides with, for example, steroid signal modulators and/or redox effectors. Additionally, cardiac glycosides may be combined with radiation therapy, taking advantage of the radiosensitizing effect that many cardiac glycosides have.

In certain embodiments, the cardiac glycoside comprises a steroid core with either a pyrone substituent at C17 (the “bufadienolides form”) or a butyrolactone substituent at C17 (the “cardenolide” form).

In certain embodiments, the cardiac glycoside is represented by the general formula:

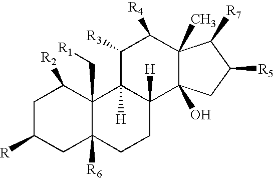

wherein

R represents a glycoside of 1 to 6 sugar residues;

R₁ represents hydrogen, —OH or ═O;

R₂, R₃, R₄, R₅, and R₆ each independently represents hydrogen or —OH;

R₇ represents

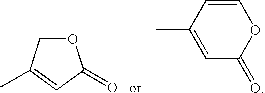

In certain preferred embodiments, the sugar residues are selected from L-rhamnose, D-glucose, D-digitoxose, D-digitalose, D-digginose, D-sarmentose, L-vallarose, and D-fructose. In certain embodiments, these sugars are in the β-conformation. The sugar residues may be acetylated, e.g., to effect the lipophilic character and the kinetics of the entire glycoside. In certain preferred embodiments, the glycoside is 1-4 sugar residues in length.

In certain embodiments, the cardiac glycoside is selected from digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocyrnarin, digoxin (CP 4072), strophanthidin oxime, strophanthidin semicarbazone, strophanthidinic acid lactone acetate, ernicyrnarin, sannentoside D, sarverogenin, sarmentoside A, sarmentogenin, or a pharmaceutically acceptable salt, ester, amide, or prodrug thereof.

In certain preferred embodiments, the cardiac glycoside is ouabain or proscillaridin.

B. Exemplary Treatment Methods Using Na⁺/K⁺-ATPase Inhibitors

The subject Na⁺/K⁺-ATPase inhibitor, such as a cardiac glycoside agonists (e.g. ouabain or proscillaridin, etc.) may be used in the treatment or prevention of a number of neoplastic and non-neoplastic angiogenesis diseases in a mammalian patient (human or non-human), wherein the patient has, or is at risk for developing such diseases.

Neoplastic diseases include: tumor growth, hemangioma, meningioma, solid tumors, leukemia, neovascular glaucoma, angiofibroma, pyogenic granuloma, scleroderma, trachoma, and metastasis thereof.

Non-neoplastic angiogenesis diseases include, but are not limited to: retinal neovascularization, diabetic retinopathy, retinopathy of prematurity (ROP), endometriosis, macular degeneration, age-related macular degeneration (ARMD), psoriasis, arthritis, rheumatoid arthritis (RA), atherosclerosis, hemangioma, Kaposi's sarcoma, thyroid hyperplasia, Grave's disease, arterioyenous malformations (AVM), vascular restenosis, dermatitis, hemophilic joints, hypertrophic scars, synovitis, vascular adhesions, and other inflammatory diseases.

In certain other embodiments, the mammalian patient has, or is at risk for developing abnormal proliferation of fibrovascular tissue, acne rosacea, acquired immune deficiency syndrome, artery occlusion, atopic keratitis, bacterial ulcers, Bechets disease, blood borne tumors, carotid obstructive disease, chemical burns, choroidal neovascularization, chronic inflammation, chronic retinal detachment, chronic uveitis, chronic vitritis, contact lens overwear, corneal graft rejection, corneal neovascularization, corneal graft neovascularization, Crohn's disease, Eales disease, epidemic keratocon junctivitis, fungal ulcers, Herpes simplex infections, Herpes zoster infections, hyperviscosity syndromes, Kaposi's sarcoma, leukemia, lipid degeneration, Lyme's disease, marginal keratolysis, Mooren ulcer, Mycobacteria infections other than leprosy, myopia, ocular neovascular disease, optic pits, Osler-Weber syndrome (Osler-Weber-Rendu, osteoarthritis, Pagets disease, pars planitis, pemphigoid, phylectenulosis, polyarteritis, post-laser complications, protozoan infections, pseudoxanthoma elasticum, pterygium keratitis sicca, radial keratotomy, retinal neovascularization, retinopathy of prematurity, retrolental fibroplasias, sarcoid, scleritis, sickle cell anemia, Sogrens syndrome, solid tumors, Stargarts disease, Steven's Johnson disease, superior limbic keratitis, syphilis, systemic lupus, Terrien's marginal degeneration, toxoplasmosis, trauma, tumors of Ewing sarcoma, tumors of neuroblastoma, tumors of osteosarcoma, tumors of retinoblastoma, tumors of rhabdomyosarcoma, ulceritive colitis, vein occlusion, Vitamin A deficiency or Wegeners sarcoidosis.

In certain other embodiments, the mammalian patient has, or is at risk for developing diabetes; parasitic disease; abnormal wound healing; hypertrophy following surgery, burns, injury or trauma; inhibition of hair growth; inhibition of ovulation and corpus luteum formation; inhibition of implantation or inhibition of embryo development in the uterus.

In certain other embodiments, the mammalian patient has, or is at risk for developing graft rejection, lung inflammation, nephrotic syndrome, preeclampsia, edema associated with brain tumors, ascites associated with malignancies, Meigs' syndrome, pericardial effusion, pericarditis or pleural effusion.

In fact, dozens of diseases are associated with overactive blood vessel growth, and thus are good candidates for antiangiogenesis therapy using the subject Na⁺/K⁺-ATPase inhibitor (e.g. cardiac glycoside agonists). For example, angiogenesis associated with psoriasis contributes to the increasing turnover of epithelial cells and puritic plaques; it contributes to Crohn disease by providing a way for inflammatory cells to enter sites of injury; it is part of the pannus, the excessive folds of inflamed tissue, in rheumatoid arthritis; and it causes intraperitoneal bleeding in endometriosis. Atherosclerosis, obesity, diabetes, and even Alzheimer's Disease also depend on angiogenesis.

The following sections describe various disease conditions that can be treated/prevented using the subject Na⁺/K⁺-ATPase inhibitor.

Diabetic Retinopathy

In one aspect of the invention, the subject Na⁺/K⁺-ATPase inhibitors, such as the cardiac glycosides (e.g. ouabain or proscillaridin, etc.), may be delivered/administered to retinal tissue to down-regulate VEGF expression therein. It is known that VEGF causes retinal neovascularization in animals including human beings suffering from diabetic retinopathy. Diabetic retinopathy is a common microvascular complication in patients with type 1 diabetes. The progression of background retinopathy to proliferative retinopathy leads to visual impairment through bleeding or retinal detachment by accompanying fibrous tissues.

Experiments in animal models with induced ocular neovascularization show that VEGF is up-regulated several folds before the formation of new blood vessels, and that blocking its action inhibits retinal neovascularization. Also, increased vascular permeability is a characteristic sign of early stages (background retinopanty) of diabetic retinopathy, and VEGF is up-regulated during this stage. Retinal digest preparations from diabetic animals and humans show scattered capillary occlusions which is a stimulus for increased vascular permeability. VEGF is such a vascular permeability factor.

Diabetic rat model of experimental retinopathy may be used to screen candidate anti-angiogenesis factor, and to test and/or verify the efficacy of a candidate anti-angiogenesis factor in the retinal tissue. Such diabetic rat model of retinopathy is known to one skilled in the art. For example, chronic hyperglycemia can be induced in 4-6 week old Wistar rats by intravenous injection of 60-65 mg/kg body weight streptozotocin. Diabetes can be monitored consecutively by taking body weight and blood glucose levels into consideration.

To illustrate, when these rats reach, for example, a body weight of about 330 g and their blood glucose levels of 25 mmol/l, the subject Na⁺/K⁺-ATPase inhibitor, such as a cardiac glycoside (e.g. ouabain or proscillaridin, etc.) can be administered to the retinal tissue at 1 to 2 week intervals. The age-matched nondiabetic rats are used as controls. VEGF levels can be monitored in the retinal tissues of diabetic and control rats at regular intervals of 7 to 14 days, by any of the suitable techniques such as in situ hybridization for VEGF, immunoreactivity, immunohistochemistry and western blot analysis. For example, retinal protein extracts can be performed to confirm the relative decrease in VEGF protein levels in retinal tissue. The treatments are continued until VEGF levels in the retinal extracts are similar to that in nondiabetic rats. Quantitation of cellular capillaries can also be performed in diabetic rats and compared to that of the controls. Thus therapies using the subject Na⁺/K⁺-ATPase inhibitor, such as a cardiac glycoside (e.g. ouabain or proscillaridin, etc.) provide effective anti-VEGF strategy in diabetic retinopathy.

Choroidal Neovascularization

In another aspect, the methods, reagents, and pharmaceutical compositions of the present invention can be used to inhibit choroidal neovascularization (CNV). CNV is a serious complication of age related macular degeneration and it is characterized by the growth of new blood vessels from the choroid, through the Buch's membrane into the subretinal space. This ultimately leads to the formation of choroidal neovascular membranes from which blood and serum may leak, causing vision loss. At present age-related macular degeneration is clinically difficult to treat.

It is known that VEGF is a causative agent in a variety of ocular angiogenic diseases including age-related macular degeneration. For example, it has been shown that the overexpression of VEGF in retinal pigment epithelial cells is sufficient to induce CNV (Spilsbiry et al. Am J Pathol 1257:135-144, 2000).

The animal models of choroidal neovascularization in the subretinal space are well known in the art (Tobe et al. J. Jpn Ophthalmol Soc 98:837-845, 1994; Shen et al., Br J Ophthamomol 82:1062-1071, 1998). For example, a rat with CNV can be administered with a subject Na⁺/K⁺-ATPase inhibitor, such as a cardiac glycoside (e.g. ouabain or proscillaridin, etc.), with or without other anti-angiogenesis therapeutic agents. Such a treatment protocol may be used to determine whether it is sufficient to down-regulate VEGF expression and inhibit CNV in the rat.

Briefly, the CNV rats can be used for subretinal administration of the subject Na⁺/K⁺-ATPase inhibitors (with or without other therapeutic agents). The animals are anesthetized, for example, by a mixture of ketamine and xylazine administered intramuscularly. The eyes can be further treated with topical amethocaine drops and the pupils dilated with 1% tropicamide and 2.5% phenylephrine hydrochloride drops. The conjunctiva can be cut close to the limbus to expose the sclera. A 32 gauge needle is then passed through this hole in a tangential direction under an operating microscope, to deliver the agents to the subretinal space. Immediately after the subretinal injection a circular bleb is usually observed under the operating microscope. The success of each subretinal injection is further confirmed by the observation of a partial retina detachment as seen by indirect ophthalmoscopy. The needle is kept in the subretinal space for 1 minute, withdrawn gently, and antibiotic ointment applied to the wound site.

VEGF levels can be determined by VEGF mRNA expression in RPE cells. In addition, to determine whether administering an agent in the RPE has down-regulated VEGF, which VEGF expression would otherwise have a vasopermeabilty effect on blood vessels, fluorescein angiograms can be used to detect vascular leakage. Fluorescein angiography in the context of CNV is well known in the art. For example, fluorescein angiograms 5-10 days post-subretinal injection of the agent(s) can be performed to determine areas of vascular leakage.

Thus the subject Na⁺/K⁺-ATPase inhibitors, such as a cardiac glycoside agonists (e.g. ouabain or proscillaridin, etc.) provides an ideal system for targeted anti-angiogenic gene therapy in the eye.

Rheumatoid Arthritis

In another aspect, the methods of the present invention is used to down-regulate synovial fluid (SF) VEGF levels and prevention of pathological angiogenesis in Rheumatoid arthritis (RA) patients. Rheumatoid SF neutrophils have been shown to be the predominant source of VEGF in inflamed joints in RA (see Kasama et al., Clin Exp Immunol. 2000, 121:533-538). In RA disease, synovial cells proliferate in response to inflammatory stimuli, which leads to the formation of a very aggressive invasive tissue, the rheumatoid pannus.

In the early states of synovitis, there is the development of new vessels in the synovium, which deliver nutrients, oxygen, and cells to the proliferating pannus. In mouse arthritis model, production of detectable levels of VEGF protein is associated with onset of clinical symptoms of arthritis. Similarly, in human RA disease, VEGF expression has been known to correlate with disease severity in patients with chronic RA (Paleolog et al. 1978) and, therefore, the prevention of the pannus induced bone erosions and loss of joint function. Inhibition of VEGF promoted angiogenesis in the synovium can provide a promising approach for the treatment of RA.

Animal models of RA are known in the art. One such animal model is the KRN/NOD mouse model (Kouskoff et al., Cell, 1996, 87:811-822). The transgenic KRN/NOD mice develop arthritis. In these animals, the disease starts between 25 and 29 days after birth with a very acute stage characterized by joint effusions and florid synovitis that spread to all joints between days 27 and 36. The nontransgenic KRN/NOD mice remain in good condition with no signs of arthritis during this period.

The down-regulation of VEGF activity in vivo may be achieved by administration of the subject Na⁺/K⁺-ATPase inhibitor, such as a cardiac glycoside (e.g. ouabain or proscillaridin, etc.), with or without other anti-angiogenesis factors. Such agents are delivered together to synovial joints in order to transduce synovial cells, including leucocytes. The agents can be delivered, for example, by direct injection into the synovium. For example, the agents can be administered on the day of arthritis onset and every other day for 14 days. Control animals receive the carrier solvents only, but with the same treatment regimen. Throughout the disease duration the animals are scored for clinical symptoms of arthritis. In the control animals, arthritis development is expected to be unaltered. As part of the assessment, arthritis is quantified by measuring the thickness of each paw, for example, with a caliper-square. Then an arthritis index is calculated for each animal as the sum of the measures of the paws.

Some of the joints into which the subject therapeutic agents can be delivered for treatment, and analysis after the treatment are wrist, ankle, knee, shoulder, elbow, metacarpophalangeal, metatarsophalangeal and hip joints. Tendon ruptures, synovial membranes invaded by the inflammatory materials, articular space filled with inflammatory materials, severe destructive lesions of the tarsal and carpal joints, panus proliferation and invasion, very intense bone lesions in terms of bone or cartilage destruction, fibrosis and fusion are some of the features of arthritis which are seen in the control RA animals but should be absent or should be seen with reduced severity in treated animals. In other words, administration of the subject therapeutic agents reduce the clinical score as well as the extent of synovitis and joint destruction, which is indicative of a suppression of the formation of the pannus. Since blood vessels are required to nourish and maintain the pannus, inhibiting angiogenesis and snyovial mass is almost certainly associated with a decrease in the total number of blood vessels.

The acute-phase response, as measured by C-reactive protein (CRP), is a marker for RA disease activity and is commonly used in clinical practice to monitor RA disease activity. Elevated levels of CRP are generally indicative of disease progression and lack of improvement under therapy. It is known that serum CRP levels significantly correlate with both cell-associated and free VEGF in SF, thereby providing a useful method for monitoring the disease activity of RA. Also, serum VEGF concentration has been reposted to correlate with serum CRP level and RA activity correlates with serum concentration of CRP (Harada et al., Scand J Rheumatol, 1998 27:377-380), and therefore serum VEGF level represents the disease activity of RA.

Hemangioma

Hemangiomas are angiogenic diseases, characterized by the proliferation of capillary endothelium with accumulation of mast cells, fibroblasts and macrophages. They represent the most frequent tumors of infancy, occurring more frequently in females than males (3:1 ratio). Hemangiomas are characterized by rapid neonatal growth (proliferating phase). By the age of 6 to 10 months, the hemangioma's growth rate becomes proportional to the growth rate of the child, followed by a very slow regression for the next 5 to 8 years (involuting phase). Most hemangiomas occur as single tumors whereas about 20% of the affected infants have multiple tumors, which may appear at any body site. Approximately 5% produce life-, sight-, or limb-threatening complications, with high mortality rates. Several immunohistochemical studies have provided insight into the histopathology of these lesions. In particular, proliferating hemangiomas express high levels of proliferating cell nuclear antigen (PCNA, a marker for cells in the S phase), type IV collagenase, VEGF and FGF-2. During the involuting phase of hemangiomas, expression of these angiogenic factors decreases. Furthermore, urinary levels of FGF-2 are elevated during the proliferating phase of hemangioma, but become normal during involution or after therapy with IFN-a.

Psoriasis and Kaposi's Sarcoma

Psoriasis and Kaposi's sarcoma are proliferative disorders of the skin. Hypervascular psoriatic lesions express high levels of the angiogenic inducer IL-8, whereas the expression of the endogenous inhibitor TSP-1 is decreased. Kaposi's sarcoma (KS) is the most common tumor associated with human immunodeficiency virus (HIV) infection and is in this setting almost always associated with human herpes virus 8 (HHV-8) infection. Typical features of KS are proliferating spindle-shaped cells, considered to be the tumor cells and endothelial cells forming blood vessels. KS is a cytokine-mediated disease, highly responsive to different inflammatory mediators like IL-1b, TNF-a and IFN-g and angiogenic factors. In particular, FGF-2 was found to synergize with HIV-tat to promote angiogenesis and KS development. Finally, growth of KS, both in vitro and in vivo, could be blocked by an antisense oligonucleotide targeting FGF-2.

Atherosclerosis

Angiogenesis also contributes to atherosclerosis, a major cause of death of Western populations. Atherosclerosis is the main cause of heart attack. The walls of the coronary artery are normally free of microvessels except in the atherosclerotic plaques, where there are dense networks of capillaries, known as the vasa vasorum. These fragile microvessels can cause hemorrhages, leading to blood clotting, with a subsequent decreased blood flow to the heart muscle and heart attack.

Thus, the present invention provides methods for down-regulating angiogenetic factors to inhibit angiogenesis in vivo in treating/preventing diseases described above, by delivering a Na⁺/K⁺-ATPase inhibitor, such as a cardiac glycoside agonist (e.g. ouabain or proscillaridin, etc.), with or without other anti-angiogenesis factors.

Alzheimer's Disease (AD)

Alzheimer's disease (AD), characterized by impairments in cognition and memory, is clearly associated with the slow accumulation of amyloid β peptides (AβPs) in the central nervous system (Selkoe, Physiol. Rev. 81: 741-766, 2001; Small et al., Nat. Rev. Neurosci. 2: 595-598, 2001). AβPs are generated via amyloidogenic processing of amyloid precursor protein (APP) by β- and γ-secretases, and recent evidence suggests that γ-secretase activity requires the formation of a complex between presenilin, nicastrin, APH-1 and pen-2 (Edbauer et al., Nat. Cell Biol. 5: 486-488, 2003). Disruption of Ca²⁺ homeostasis has been strongly implicated in the neurodegeneration of AD; indeed, increased Ca²⁺-dependent protease activity occurs in association with degenerating neurones in AD brain tissue (Nixon et al., Ann. N Y Acad. Sci. 747: 77-91, 1994), and AβPs perturb Ca²⁺ homeostasis, rendering cells susceptible to excitotoxic damage (Mattson et al., J. Neurosci. 12: 376-389, 1992). Presenilin mutations are known to have effects on cellular Ca²⁺ homeostasis (Mattson et al., Trends Neurosci. 23, 222-229, 2000), and familial AD (FAD)-related mutations of presenilin-1 (PS-1) can alter inositol triphosphate-coupled intracellular Ca²⁺ stores as well as Ca²⁺ influx pathways (Leissring et al., J. Cell Biol. 149, 793-798, 2000; Mattson et al., Trends Neurosci. 23, 222-229, 2000; Yoo et al., Neuron 27, 561-572, 2000). This may contribute to neurodegeneration, since disruption of Ca²⁺ homeostasis is an important mechanism underlying such loss of neurones (Chan et al., J. Biol. Chem. 275, 18195-18200, 2000; Mattson et al., J. Neurosci. 20, 1358-1364, 2000; Yoo et al., supra).

Periods of cerebral hypoxia or ischaemia can increase the incidence of AD (Tatemichi et al., Neurology 44, 1885-1891, 1994; Kokmen et al., Neurology 46, 154-159, 1996), and APP expression is elevated following mild and severe brain ischaemia (Kogure and Kato, Stroke 24, 2121-2127, 1993). Since the non-amyloidogenic cleavage product of APP (sAPPa) is neuroprotective (Mattson, Physiol. Rev. 77, 1081-1132, 1997; Selkoe, Physiol. Rev. 81, 741-766, 2001), increased expression during hypoxia could be considered a protective mechanism against ischaemia. However, increased APP levels would also provide an increased substrate for AβP formation. It was previously shown that AβP formation is increased following hypoxia in PC12 cells (Taylor et al., J. Biol. Chem. 274, 31217-31222, 1999; Green et al., J. Physiol. 541, 1013-1023, 2002). Furthermore, prolonged hypoxia potentiates bradykinin (BK)-induced Ca²⁺ release from intracellular stores in rat type I cortical astrocytes. This was due to dysfunction of mitochondria and plasmalemmal Na⁺/Ca²⁺ exchanger (NCX; Smith et al., J. Biol. Chem. 278, 4875-4881, 2003). Peers et al. (Biol Chem. 385(3-4): 285-9, 2004) report that sustained central hypoxia predisposes individuals to dementias such as Alzheimer's disease, in which cells are destroyed in part by disruption of Ca²⁺ homeostasis. Moreover, hypoxia increases the levels of presenilin-1, a major component of a key enzyme involved in Alzheimer's disease. Thus there is established link between periods of hypoxia and the development of AD.

Thus, the present invention provides methods for inhibiting the onset and/or development of AD by inhibiting hopoxia-induced effects using the subject Na⁺/K⁺-ATPase inhibitors, such as the cardiac glycosides (e.g. ouabain or proscillaridin, etc.). Such methods and reagents can be used with other AD treatment methods and drugs in treating/preventing AD.

The U.S. Food and Drug Administration (FDA) has approved two classes of drugs to treat cognitive symptoms of Alzheimer's disease. The first Alzheimer medications to be approved were cholinesterase inhibitors. Three of these drugs are commonly prescribed—donepezil (Aricept®); rivastigmine (Exelon®); and galantamine (Reminyl®). Tacrine (Cognex®), the first cholinesterase inhibitor, was approved in 1993 but is disfavored today because of associated side effects, including possible liver damage. Nonetheless, it is still an option.

Memantine (Namenda®) is a drug approved in October 2003 by the FDA for treatment of moderate to severe Alzheimer's disease.

Vitamin E supplements are often prescribed as a treatment for Alzheimer's disease, because research has shown that taking vitamin E supplements may offer some benefit to people with Alzheimer's.

Several herbal remedies and other dietary supplements are promoted as effective treatments for Alzheimer's disease and related disorders. Such alternative treatment includes the use of Coenzyme Q10 (or its synthetic version idebenone), Ginkgo biloba, Huperzine A, Phosphatidylserine, and Coral calcium, etc.

All these treatment options may be used with the subject compound in treating/preventing Alzheimer's Disease.

Obesity

It is thought that adipose tissue exhibits angiogenic activity and also that adipose tissue mass can be regulated via the vasculature, but the relationship between adipocyte differentiation and neovascularization during de novo fat tissue formation has been unclear. However, a recent research article shows that there is reciprocal paracrine regulation of adipogenesis and angiogenesis, and that a blockade of vascular endothelial growth factor (VEGF) signaling can inhibit in vivo adipose tissue formation (D. Fukumura et al., Circulation Research, DOI:10.1161/01, Oct. 3, 2003).

Dai Fukumura et al. implanted preadipocytes into chambers beneath the skin of immune-deficient mice, thus developing a model to visualize noninvasively and in real time both angiogenesis and adipogenesis using intravital microscopy. The authors observed that inhibition of adipocyte differentiation by transfection of preadipocytes with an inactivated form of a protein required for fat cells to mature not only abrogated fat tissue formation, but also reduced angiogenesis. They also showed that that inhibition of angiogenesis by vascular endothelial growth factor receptor 2 (VEGFR2)-blocking antibody not only reduced angiogenesis and tissue growth, but also inhibited preadipocyte differentiation, partially as a result of paracrine interaction between endothelial cells and preadipocytes mediated by VEGF-VEGFR2 signaling in the endothelial cells.

Thus, the present invention provides methods for down-regulating angiogenetic factors to inhibit angiogenesis in vivo in treating/preventing obesity, by delivering a Na⁺/K⁺-ATPase inhibitor, such as a cardiac glycoside agonist (e.g. ouabain or proscillaridin, etc.), with or without other anti-angiogenesis factors.

Inflammatory Diseases

Angiogenesis and enhanced microvascular permeability are hallmarks of a large number of inflammatory diseases. There is considerable evidence to suggest that angiogenesis and chronic inflammation are closely linked (Jackson et al., FASEB J 11: 457-465, 1997). Angiogenic blood vessels at the site of inflammation are enlarged and hyperpermeable to maintain the blood flow and to meet the increased metabolic demands of the tissue (Jackson et al., Supra). Several proangiogenic factors, including vascular endothelial growth factor (VEGF) (Detmar, J. Dermatol. Sci. 24(suppl 1): S78-S84, 2000; Brown et al., J. Invest. Dermatol. 104: 744-749, 1995; Fava et al., J. Exp. Med. 180: 341-346, 1994) and members of the CXC-chemokine family (Schroder and Mochizuki, Biol. Chem. 380: 889-896, 1999; Strieter et al., Shock. 4: 155-160, 1995) have been found to be up-regulated during inflammation. While not wishing to be bound by any particular theory, inflammation may induce local hypoxia response and promote angiogenesis through, for example, VEGF and other factors.

Thus, the present invention provides methods for down-regulating angiogenetic factors to inhibit angiogenesis in vivo in treating/preventing inflammatory diseases, by delivering a Na⁺/K⁺-ATPase inhibitor, such as a cardiac glycoside agonist (e.g. ouabain or proscillaridin, etc.), with or without other anti-angiogenesis factors.

Cataract Surgery

In normal lenses, immunoreactivity against bufalin and ouabain-like factor is sevenfold to 30-fold higher in the capsular epithelial layer than in the lens fiber region (Lichtstein et al., (2000) Involvement of Na⁺,K⁺-ATPase inhibitors in cataract formation. In Na/K-ATPase and Related ATPases (Taniguchi, K. & Haya, S., eds), Elsevier Science, Amsterdam). In human cataractous lenses, the concentration of the sodium pump inhibitor was much higher than in normal lenses. Hence, it was isolated from cataractous lenses and identified as 19-norbufalin and its Thr-Gly-Ala tripeptide derivative (Lichtstein et al., Eur. J. Biochem. 216: 261-268, 1993). Cardiac glycosides alter the osmotic balance of lenses and induce cataract formation by crystalline degradation and protein leakage that initiate opacity. On the other hand, cataract surgery will remove such sources of cardiac glycoside agonists, thus may also lose the local inhibitory effect to undesirable angiogenesis in the eye. Patients after cataract surgery may therefore be more vulnerable to conditions associated with abnormal angiogenesis. The subject compounds (or in combination with other anti-angiogenesis factors) may help to prevent/alleviate the risk or symptoms of such situations.

In addition, different diseases may interact with one another and cause complications. For example, hypertension tends to make some forms of macular degeneration worse, particularly in the “wet” form where the retinal tissues are invaded by new blood vessels. The medications used to treat hypertension have not been shown to have any direct effect on macular degeneration, but they may slow progression of the disease by reducing hypertension. Also, any type of smoking or exposure to tobacco smoke can accelerate the development of the “wet” type of macular degeneration. Thus it is contemplated that the compounds of the invention may be used in combination therapy with pharmaceutical compositions for treating those other diseases/conditions, such as hypertension or smoking.

C. Exemplary Cardiac Glycoside Antagonists

WO0125281A1 describes a monoclonal antibody or antigen binding fragment thereof having binding specificity for ouabain, wherein the antibody or antigen binding fragment does not crossreact with digoxin. In certain embodiments, the anti-ouabain monoclonal antibody binds ouabain with an affinity of at least about 10⁻⁷ M, preferably 10⁻⁸ M, and more preferably 10⁻⁹ M. These monoclonal antibodies are useful when specific inhibition of ouabain or ouabain-like cardiac glycosides (but not digoxin or digoxin-like cardiac glycosides) are desired.

U.S. Pat. No. 5,164,296 describes a polyclonal antibody directed against ouabain. In addition, a mAb to Ouabain showing a high degree of cross-reactivity with digoxin (Dig) is described in WO 01/25281. Other cardiac glycoside (e.g. ouabain) antibodies are described in U.S. Pat. No. 5,844,091, U.S. Pat. No. 5,656,434, U.S. Pat. No. 5,429,928, etc. All contents incororated herein by reference. These antibodies or functional fragments thereof may be useful to treat conditions associated with excessive levels of ouabain and/or digoxin (and there analogs).

Additional antibodies against any cardiac glycosides of the subject application may be produced using routine monoclonal/polyclonal antibody production and screening methods, such as those described in Antibodies: A Laboratory Manual, Eds. Harlow and Lane, Cold Spring Harbor Laboratory Press; (Dec. 1, 1988). Depending on specific needs, these antibodies or fragments thereof (Fab, Fd, scFv, F(ab)₂, etc.) may be specific for just one or a few of cardiac glycosides, or of generic specificity for most cardiac glycosides, as exemplified above.

Another class of cardiac glycoside antagonists is described by Ferrari et al. (J Pharmacol Exp Ther. 1998 April;285(1):83-94). It was disclosed that 17beta-(3-furyl)-5beta-androstane-3beta, 14beta, 17alpha-triol (or PST 2238) displaced ouabain from its binding sites on purified sodium, potassium ATPase enzyme (Na-K ATPase) (IC₅₀ 1.7×10⁻⁶ M) without interacting with other receptors involved in blood pressure regulation or hormonal control. The ouabain-dependent increase in the Na-K ATPase pump rate was abolished by PST 2238 at concentrations from 10⁻¹⁴ to 10⁻⁹ M. Thus PST 2238 represents the prototype of a new class of very potent compounds that antagonizes ouabain function, and can be used in the subject methods of treating conditions associated with excessive amount of cardiac glycosides, such as depression, and all the symptoms of digitalis poisoning.

EP0576915A2 and EP0583578A2 (entire contents incorporated herein by reference) describe in detail about the synthesis of the PST 2238 family of compounds (e.g. 17-(3-furyl) and 17-(4-pyridazinyl)-5beta,14beta-androstane derivatives, etc.), and a series of biological assays that can be used to determine the function of the synthesized compounds. The assays include: a) displacement of the specific ³H-ouabain binding from the Na⁺,K⁺-ATPase receptor purified according to Jorghensen (Jorghensen P., BBA, 1974, 356, 36) and Erdmann (Erdmann E. et al., Arzneim. Forsh., 1984, 34 (II), 1314); b) inhibition of the activity of ouabain on the purified Na⁺,K⁺-ATPase measured as % of hydrolysis of ³²P-ATP in presence and in absence of the tested compound (Mall F. et al., Biochem. Pharmacol., 1984, 33, 47). The ability of these compounds to lower blood pressure in adult hypertensive MHS rats was tested by the following method: systolic blood pressure (SBP) and heart rate (HR) were measured by an indirect tail-cuff method in three-month old hypertensive MHS rats before beginning treatment (basal values). The rats were then subdivided in two groups of 7 animals each, one receiving the compound and the other, the control group, receiving only the vehicle. The compound, suspended in METHOCEL® 0.5% (w/v), for ten days, was administered daily by mouth. SBP and HR were measured daily 6 and 24 hours after the treatment. When ten-day treatment washout had been under way for at least two days, whether the treatment maintains SBP low or re-establish the basal values was verified.

Other compounds that may be used as antagonists of cardiac glycosides are disclosed in EP0659761A2, EP0688786A1, EP0714908A2, and EP0825177A1 (entire contents incorporated herein by reference).

Compounds synthesized and verified using these methods may be effectively used in the subject invention as antagonists of cardiac glycosides.

D. Treatment of Conditions Associated with Excessive Cardiac Glycosides

Digitalis poisoning is largely caused by excessive cardiac glycosides. Significant toxicity usually is resultant of depression and a suicide attempt.

More than 200 naturally occurring cardiac glycosides have been identified. These bind to a site on the cell membrane, producing reversible inhibition of the sodium (Na⁺)-potassium (K⁺)-adenosine triphosphatase (ATPase) pump, which causes increased intracellular sodium and decreased intracellular potassium. In myocytes, elevated intracellular sodium concentrations produce increased intracellular calcium concentrations via a Na⁺-calcium (Ca⁺⁺)-exchanger. Excessive intracellular calcium is concentrated in sarcoplasmic reticulum and released, in excess, with depolarization. Release of excessive calcium results in enhanced cardiac contractions, which are delayed after depolarizations and manifest clinically as after contractions, such as premature ventricular contractions (PVCs). Cardiac glycosides also have vagotonic effects, resulting in bradycardia and heart blocks. Inhibition of Na⁺/K⁺-ATPase in skeletal muscle results in increased extracellular potassium and contributes to hyperkalemia.

Cardiac glycosides primarily affect cardiovascular, neurologic, and gastrointestinal systems. Of these, effects on the cardiac system are most significant. The pathophysiology that produces cardiotoxicity involves prolonging refractory period in atrioventricular (AV) node, shortening refractory periods in atria and ventricles, and decreasing resting membrane potential (increased excitability). At therapeutic doses, cardiac glycosides also may increase inotropy. Any dysrhythmia characterized by both increased automaticity and depressed conduction is suggestive of cardiac glycoside toxicity.

Sinus rhythm with PVCs is the most common rhythm associated with digitalis toxicity. Dysrhythmias often associated with cardiac glycoside toxicity include bradydysrhythmias, sinus bradycardia with all types of AV nodal block, junctional rhythms, and sinus arrest. Dysrhythmias characterized by increased automaticity and conduction blockade, which combined are highly suggestive of cardiac toxicity. These dysrhythmias include the following:
Tachydysrhythmias, such as atrial tachycardia with block
Junctional tachycardia
Ventricular tachycardia
Ventricular fibrillation
Paroxysmal atrial tachycardia with block
Bidirectional ventricular tachycardia

Various dysrhythmias may alternate or progress rapidly into life-threatening rhythms, such as ventricular tachycardia.

Thus symptoms of digitalis poisoning include one or more of: fatigue, visual symptoms, muscle weakness to vertigo, nausea, anorexia, psychic complaints, abdominal pain, dizziness, abnormal dreams, headache, diarrhea, vomiting, syncope, lethargy, seizures, impaired memory, confusion, disorientation, delusions, depression, and delirium, etc.), hypokalemia, bradydysrhythmias and AV blocks, ventricular tachydysrhythmias, atrial tachycardia with block, non-paroxysmal junctional tachycardia, atrioventricular conduction disturbances, first-degree block, sinus impulse formation disturbances, sinus bradycardia, sinus arrest, sinoatrial block, second-degree atrioventricular block, third-degree atrioventricular block, non-Paroxysmal atrial tachycardia with block, non-paroxysmal junctional tachycardia, atrioventricular dissociation, second-degree atrioventricular block (Type I), premature ventricular contractions, ventricular bigeminy, ventricular tachycardia, bidirectional ventricular tachycardia, ventricular fibrillation.

GI symptoms associated with digitalis toxicity are usually the first symptoms to evolve. These symptoms seem nonspecific, and include nausea, vomiting, abdominal pain, diarrhea, and anorexia. Neurological symptoms often include giddiness, headache, dizziness, fatigue, weakness, numbness (especially of tongue and lips), hallucinations, altered mental status (e.g., disorientation, confusion, drowsiness, lethargy), and seizures. Findings may include an altered level of consciousness, hypotonia, hyporeflexia, dysarthria, ataxia, horizontal nystagmus, and generalized seizures. Visual effects include blurred vision, scotomas, and flashes of light. Abnormal color perceptions of yellow or yellow-green halos (e.g., xanthopsia) may occur. Cardiac symptoms including palpitations, fluttering in chest, chest pressure or shortness-of-breath, lightheadedness, dizziness, faintness, and sensation of irregular heartbeat may be noted.

The level of cardiac glycosides in the serum of patient with digitalis poisoning varies, but is usually more than the therapeutic range of cardiac glycosides, such as 0.5 to 2 ng/ml for digoxin. Typically, the serum level of cardiac glycosides in the intoxicated patients is about 2-5 ng/ml, or more than about 3 ng/ml. However, in certain patients, the serum cardiac glycosides may be as low as about 1 ng/ml or less, and the patients still manifest symptoms of digitalis toxicity. The antagonists of the cardiac glycosides are contemplated to be effective in these patients too.

In certain embodiments, the antagonists of cardiac glycosides may be used with other depression medicine/drug in the same patient, either simultaneously or sequentially, over one dose or many doses over a period of time. The depression drugs that may be used with the antagonists of cardiac glycoside include:
The SSRI's (Selective Serotonin Reuptake Inhibitors), which increase the brain's level of serotonin, thus improving mood. SSRI's include: Celexa (citalopram); Lexapro (escitalopram HBr); Luvox (fluvoxamine); Paxil, Paxil CR (paroxetine); Prozac, Prozac Weekly (fluoxetine); Zoloft (sertraline).
Tricyclic Antidepressants: Amitriptyline; Desipramine; Nortriptyline.
the MAOI's (monoamine oxidase Inhibitors): Nardil (phenelzine); Pamate (tranylcypromine).
others: Cymbalta (Duloxetine; Drug Family: serotonin and norepinephrine uptake inhibitor); Effexor, Effexor XR (venlafaxine; Drug Family: serotonin and norepinephrine uptake inhibitor); Remeron (mirtazepine); Serzone (nefazodone); Trazodone; Wellbutrin, Wellbutrin SR, Wellbiutrin XL (bupropion).

The effect of most of these antidepressants seems to be at its best when the drugs are used in combination with counseling, such as seeing a psychiatrist, psychologist, social worker or other health professional on a regular basis.

E. Administration

The subject Na⁺/K⁺-ATPase inhibitor, e.g. cardiac glycoside, or a combination containing a subject Na⁺/K⁺-ATPase inhibitor (e.g. cardiac glycoside), or an antagonist of cardiac glycosides may be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out. In another instance the topical administration may be ophthalmic, with direct application of the therapeutic composition to the eye.

In a preferred embodiment, the subject Na⁺/K⁺-ATPase inhibitors (e.g. cardiac glycosides) or antagonists thereof are administered to a patient by using osmotic pumps, such as Alzet® Model 2002 osmotic pump. Osmotic pumps provides continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

To meet different therapeutic needs, ALZET's osmotic pumps are available in a variety of sizes, pumping rates, and durations. At present, at least ten different pump models are available in three sizes (corresponding to reservoir volumes of 100 μL, 200 μL and 2 mL) with delivery rates between 0.25 μL/hr and 10 μL/hr and durations between one day to four weeks.

While the pumping rate of each commercial model is fixed at manufacture, the dose of agent delivered can be adjusted by varying the concentration of agent with which each pump is filled. Provided that the animal is of sufficient size, multiple pumps may be implanted simultaneously to achieve higher delivery rates than are attainable with a single pump. For more prolonged delivery, pumps may be serially implanted with no ill effects. Alternatively, larger pumps for larger patients, including human and other non-human mammals may be custom manufactured by scaling up the smaller models.

In certain embodiments, the agents of the invention may be administered to a patient's eye in a controlled manner. There are numerous devices and methods for delivering drugs to the eye. For example, U.S. Pat. No. 6,331,313 describes various controlled-release devices which are biocompatible and can be implanted into the eye. In certain embodiments, the devices described therein have a core comprising a drug and a polymeric outer layer which is substantially impermeable to the entrance of an environmental fluid and substantially impermeable to the release of the drug during a delivery period, and drug release is effected through an orifice in the outer layer. These devices have an orifice area of less than 10% of the total surface area of the device and can be used to deliver a variety of drugs with varying degrees of solubility and or molecular weight. Methods are also provided for using these drug delivery devices. In certain embodiments, the biocompatible, implantable ocular controlled-release drug delivery device is sized for implantation within an eye for continuously delivering a drug within the eye for a period of at least several weeks. Such device comprises a polymeric outer layer that is substantially impermeable to the drug and ocular fluids, and covers a core comprising a drug that dissolves in ocular fluids, wherein the outer layer has one or more orifices through which ocular fluids may pass to contact the core and dissolve drug, and the dissolved drug may pass to the exterior of the device. The orifices in total may have an area less than one percent of the total surface area of the device, and the rate of release of the drug is determined solely by the composition of the core and the total surface area of the one or more orifices relative to the total surface area of the device.

Other ocular implant methods and devices, and related improvements for drug delivery in the eye are described in U.S. Pat. Nos. 5,824,072, 5,766,242, 5,632,984, 5, 443,505, and 5,902,598; U.S. Patent Application publications US20040175410A1, US20040151754A1, US20040022853A1, US20030203030A1, and PCT publications WO9513765A1, WO0130323A2, WO0202076A2, WO0243785A2, and WO04026106A2, to name but a few. All are incorporated herein by reference in their entirety.

In certain embodiments, the subject compounds may need to be delivered locally (such as local inflammation). In such cases, various known methods in the art may be used to achieve limited local delivery without causing undesirable systemic side effects. To just name a few, WO03066130A2 (entire contents incorporated herein by reference) discloses a transdermal delivery system including a drug formulated with a transport chaperone moiety that reversibly associates with the drug. The chaperone moiety is associated with the drug in the formulation so as to enhance transport of the drug across dermal tissue and releasing the drug after crossing said dermal tissue. The application also provides a micro-emulsion system for transdermal delivery of a drug, which system solubilizes both hydrophilic and hydrophobic components. For instance, the microemulsion can be a cosolvent system including a lipophilic solvent and an organic solvent. Exemplary cosolvents are NMP and IPM.

WO02087586A1 (entire contents incorporated herein by reference) discloses a sustained release system that includes a polymer and a prodrug having a solubility less than about 1 mg/ml dispersed in the polymer. Advantageously, the polymer is permeable to the prodrug and may be non-release rate limiting with respect to the rate of release of the prodrug from the polymer. This permits improved drug delivery within a body in the vicinity of a surgery via sustained release rate kinetics over a prolonged period of time, while not requiring complicated manufacturing processes.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams. Preferably, the route of administration is parenteral, more preferably intravenous.

F. Conjoint Therapy of Na⁺/K⁺-ATPase Inhibitors with Other Angiogenesis Inhibitors

The subject Na⁺/K⁺-ATPase inhibitors (e.g. cardiac glycosides) can also be combined with a therapeutically effective amount of one or more other molecules which negatively regulates angiogenesis which may be, but is not limited to, VEGF inhibitors such as antibodies against VEGF or antigenic epitopes thereof; soluble VEGF receptors such as Flt-1, Flk-1/KDR, Flt-4, neuropilin-1 and -2 (NP1 and NP2); TNP-470; PTK787/ZK 222584 (1-[4chloroanilino]-4-[4-pyridylmethyl]ph-thalazine succinate) (Novartis International AG, Basel, Switzerland); VEGF receptor inhibitors, such as SU5416, or antibodies against such receptors such as DC101 (ImClone Systems, Inc., NY); tyrosine kinase inhibitors; prolactin (16-KDs fragment), angiostatin (38-kD fragment of plasminogen), endostatin, basic fibroblast derived growth factor (bFGF) inhibitors such as a soluble bFGF receptor; transforming growth factor beta; interferon alfa; epidermal-derived growth factor inhibitors; platelet derived growth factor inhibitors; an integrin blocker, interleukin-12; troponin-1; 12-lipoxygenase (LOX) inhibitors, such as BHPP (N-benzyl-N-hydroxy-5-phenylpentanamide) (Nie et al. Blood 95:2304-2311); platelet factor 4; thrombospondin-1; tissue inhibitors of metalloproteases such as TIMP1 and TIMP2; transforming growth factor beta; interferon alfa; protamine; combination of heparin and steroids; and steroids such as tetrahydrocortisol; which lack gluco- and mineral-corticoid activity; angiostatin; phosphonic acid agents; anti-invasive factor; retinoic acids and derivatives thereof; paclitaxel (U.S. Pat. No. 5,994,341); interferon-inducible protein 10 and fragments and analogs of interferon-inducible protein 10; medroxyprogesterone; sulfated protamine; prednisolone acetate; herbimycin A; peptide from retinal pigment epithelial cell; sulfated polysaccharide; and phenol derivatives; isolated body wall of a sea cucumber, the isolated epithelial layer of the body-wall of the sea cucumber, the flower of the sea cucumber, their active derivatives or mixtures thereof; thalidomide and various related compounds such as thalidomide precursors, analogs, metabolites and hydrolysis products; 4 kDa glycoprotein from bovine vitreous humor; a cartilage derived factor, human interferon-alpha; ascorbic acid ethers and related compounds; sulfated polysaccharide DS 4152; and a synthetic fumagillin derivative, AGM 1470. In the preferred embodiment of the present invention, the angiogenesis inhibitor is a VEGF inhibitor. Most preferably the angiogenesis inhibitor is truncated, soluble form of a VEGF receptor.

An increasing number of anti-angiogenic compounds have been identified (see table below), many of which have been shown to hold anti-angiogenic activity in a particular assay, like the CAM. More recently, research has focused on the search for compounds with a specific effect on an individual step of the angiogenic process. As each step in the angiogenic cascade involves a great variety of enzymes, cytokines and receptors, angiogenesis presents different possible targets for therapeutic intervention. All these agents may be combined with the subject compounds to treat various angiogenesis diseases.

### Anti-Angiogenic Therapy: Compounds and Their Mechanism of Action

| | |
|---|---|
| Compound | Mechanism of action |

Inhibitors of ECM remodeling

| | |
|---|---|
| Batimastat, Marimastat, | MMP inhibitors, block endothelial |
| AG3340, Neovastat, PEX, | and tumor cell invasion |
| TIMP-1, -2, -3, -4 | |
| PAI-1, -2, uPA Ab, uPAR | uPA inhibitors, block ECM breakdown |
| Ab, Amiloride | |
| Minocycline, tetracyclines, | Collagenase inhibitors, disrupt |
| steroids, cartilage-derived | collagen synthesis and deposition |
| TIMP | |

Inhibitors of adhesion molecules

| | |
|---|---|
| avb3 Ab: LM609 and | Block EC adhesion, induce EC |
| Vitaxin, RGD containing | apoptosis |
| peptides, avb5 Ab | |
| Benzodiazapine derivatives | Antagonist of avb3 |

Inhibitors of activated endothelial cells

| | |
|---|---|
| Endogenous inhibitors: | Block EC proliferation, induce EC |
| Endostatin, Angiostatin, | apoptosis, inhibit angiogenic switch |
| aaAT | |
| IFN-a, IFN-g , IL-12, | Block EC migration and/or |
| nitric oxide synthase | proliferation |
| inhibitors, TSP-1 | |
| TNP-470, Combretastatin A4 | Block EC proliferation |
| Thalidomide | Inhibits angiogenesis in vivo |
| Linomide | Inhibits EC migration |

Inhibitors of angiogenic mediators or their receptors

| | |
|---|---|
| IFN-a , PF-4, prolactin | Inhibit FGF-2, Inhibit FGF-2-induced |
| fragment | EC proliferation |
| Suramin and analogues | Bind to various growth factors, |
| | including FGF-2, VEGF, PDGF, inhibit |
| | EC migration and proliferation |
| PPS, distamycin A | Inhibit FGF-2 activity |
| analogues, FGF-2 Ab, | |
| antisense-FGF-2 | Inhibits FGF-2 expression |
| Protamine | Binds heparin, inhibits EC migration |
| | and proliferation |
| SU5416, soluble Flt-1, | Block VEGF activity |
| dominant-negative Flk-1, | |
| VEGF receptor ribosymes, | |
| VEGF Ab | |
| Aspirin, NS-398 | COX inhibitors |
| 6-AT, 6A5BU, 7-DX | TP antagonists |

Inhibitors of EC intracellular signaling

| | |
|---|---|
| Genistein | Tyrosine kinase inhibitor, blocks |
| | uPA, EC migration and proliferation |
| Lavendustin A | Selective inhibitor of protein |
| | tyrosine kinase |
| Ang-2 | Inhibits TIE-2 |

G. Models to Study the Angiogenic Process and Screening

The invention also provides methods of screening a plurality of candidate compounds, either in in vitro or in vivo models, for their potential effects on angiogenesis. Such methods can also be used to verify and/or compare the efficacy of certain subject compounds in their abilities to inhibit angiogenesis.

Considerable insight in the molecular and cellular biology of angiogenesis has been obtained by in vitro studies using endothelial cells, isolated from either capillaries or large vessels. Most steps in the angiogenic cascade can be analyzed in vitro, including endothelial cell proliferation, migration and differentiation. Such analysis may also be used to measure IC₅₀ of certain anti-angiogenic factors in endothelial cell proliferation, migration and differentiation. The proliferation studies may be based on cell counting, thymidine incorporation, or immuno histochemical staining for cell proliferation (e.g., by measurement of PCNA) or cell death (e.g., by terminal deoxynucleotidyl transferase-mediated dUTP nick end labeling or Tunnel assay). Chemotaxis can be examined in a Boyden chamber, which consists of an upper and lower well separated by a membrane filter. Chemotactic solutions are placed in the lower well, cells are added to the top well, and after a period of incubation the cells that have migrated toward the chemotactic stimulus are counted on the lower surface of the membrane. Cell migration can also be studied by making a “wound” in a confluent cell layer and calculating the number of cells that migrate and the distance of migration of the cells from the edge of the wound. Finally, differentiation can be induced in vitro by culturing endothelial cells in different ECM components, including two- and three-dimensional fibrin clots, collagen gels and matrigel. Microvessels have also been shown to grow from rings of rat aorta embedded in a three dimensional fibrin gel.

Advantages of these in vitro systems include the possibility to control the different parameters (i.e. the spatial and temporal concentration of angiogenic mediators) involved, the ability to study individual steps in the angiogenic process, and the lower costs and efforts, as compared to in vivo experiments. However, substances with no evident chemotactic and/or mitogenic effect in vitro may nevertheless play a crucial role in angiogenesis in vivo. Therefore, the following in vivo methods are also important in angiogenesis study.

To discover and evaluate the potency of new anti-angiogenic compounds, it is advantageous to have suitable in vivo models. Classical angiogenesis assays include such well-known models as the chick chorioallantoic membrane, rabbit cornea assay, sponge implant models, matrigel plugs and conventional tumor models.

The chick chorioallantoic membrane (CAM) assay is perhaps the most widely used assay for screening purposes. The early chick embryo lacks a mature immune system and was therefore used to study tumor-induced angiogenesis. Tissue grafts were placed on the CAM through a window made in the eggshell. This caused a typical radial rearrangement of vessels towards, and a clear increase of vessels around the graft within four days after implantation. Blood vessels entering the graft were counted under a stereomicroscope. To assess the anti-angiogenic or angiogenic activity of test substances, the compounds are either prepared in slow release polymer pellets, absorbed by gelatin sponges or air-dried on plastic discs and then implanted onto the CAM. Several variants of the CAM assay including culturing of shell-less embryos in Petri dishes, and different quantification methods (i.e. measuring the rate of basement membrane biosynthesis using radio-labeled proline, counting the number of vessels under a microscope or image analysis) have been described. The CAM assay is relatively simple and inexpensive and thus suitable for large-scale screening.

The cornea presents an in vivo avascular site. Therefore, any vessels penetrating from the limbus into the corneal stroma can be identified as newly formed. To induce an angiogenic response, slow release polymer pellets (e.g. poly-2-hydroxyethyl-methacrylate (hydron) or ethylene-vinyl acetate copolymer (ELVAX)), containing an angiogenic substance (e.g. FGF-2 or VEGF) are implanted in “pockets” created in the corneal stroma of a rabbit. Also, a wide variety of tissues, cells, cell extracts and conditioned media have been examined for their effect on angiogenesis in the cornea. The vascular response can be quantified by computer image analysis after perfusion of the cornea with India ink. This method is very reliable, but technically more demanding and more expensive than the CAM assay.

Subcutaneous implantation of various artificial sponges (e.g. polyvinyl alcohol, gelatin) in animals has been used frequently to study angiogenesis in vivo. Compounds to be evaluated are either injected directly into the sponges or incorporated into ELVAX or hydron pellets, which are placed in the center of the sponge. Neovascularization of the sponges is assessed either histologically, morphometrically (vascular density), biochemically (hemoglobin content) or by measuring the blood flow rate in the vasculature of the sponge using a radioactive tracer.

Matrigel is a matrix of a mouse basement membrane neoplasm known as Engelbreth-Holm-Swarm murine sarcoma. It is a complex mixture of basement membrane proteins including laminin, collagen type IV, heparan sulfate, fibrin and growth factors, including EGF, TGF-b, PDGF and IGF-1. It was originally developed to study endothelial cell differentiation in vitro. However, matrigel-containing FGF-2 can be injected subcutaneously in mice. Matrigel is liquid at 4° C. but forms a solid gel at 37° C. that traps the growth factor to allow its slow release. After 10 days, the matrigel plugs are removed and angiogenesis is quantified histologically or morphometrically in plug sections. Matrigel is expensive, but it provides a natural environment to initiate an angiogenic response.

Numerous animal tumor models have been developed to test the anti-angiogenic and anti-cancer activity of potential drugs. In many cases, tumor cells are engrafted subcutaneously and tumor size is determined at regular time intervals. Frequently used tumor cells include C6 rat glioma, B16BL6 melanoma, LLC, and Walker 256 carcinoma. Transfection of endothelial and tumor cells with angiogenic factors has been carried out to assess the effect of overexpressing a single angiogenic factor on angiogenesis and tumor growth in vivo. Finally, the efficacy of potential anti-angiogenic agents can be evaluated on strongly vascularized tumors and in tumors of vascular origin, including polyomavirus middleT-transformed or chemically induced hemangiosarcomas, hemangioendotheliomas overexpressing FGF-2 or Kaposi's Sarcoma.

Exemplification

The following examples are for illustrative purpose only, and should in no way be construed to be limiting in any respect of the claimed invention. The exemplary cardiac glycosides used in following studies are referred to as BNC1 and BNC4.

BNC1 is ouabain or g-Strophanthin (STRODIVAL®), which has been used for treating myocardial infarction. It is a colorless crystal with predicted IC₅₀ of about 0.009-0.35 μg/mL and max. plasma concentration of about 0.03 μg/mL. According to the literature, its plasma half-life in human is about 20 hours, with a range of between 5-50 hours. Its common formulation is injectable. The typical dose for current indication (i.v.) is about 0.25 mg, up to 0.5 mg /day.

BNC4 is proscillaridin (TALUSIN®), which has been approved for treating chronic cardiac insufficiency in Europe. It is a colorless crystal with predicted IC₅₀ of about 0.002-0.008 μg/mL and max. plasma concentration of about 0.001 μg/mL. According to the literature, its plasma half-life in human is about 40 hours. Its common available formulation is a tablet of 0.25 or 0.5 mg. The typical dose for current indication (p.o.) is about 1.5 mg /day.

### EXAMPLE I

### Sentinel Line Plasmid Construction and Virus Preparation

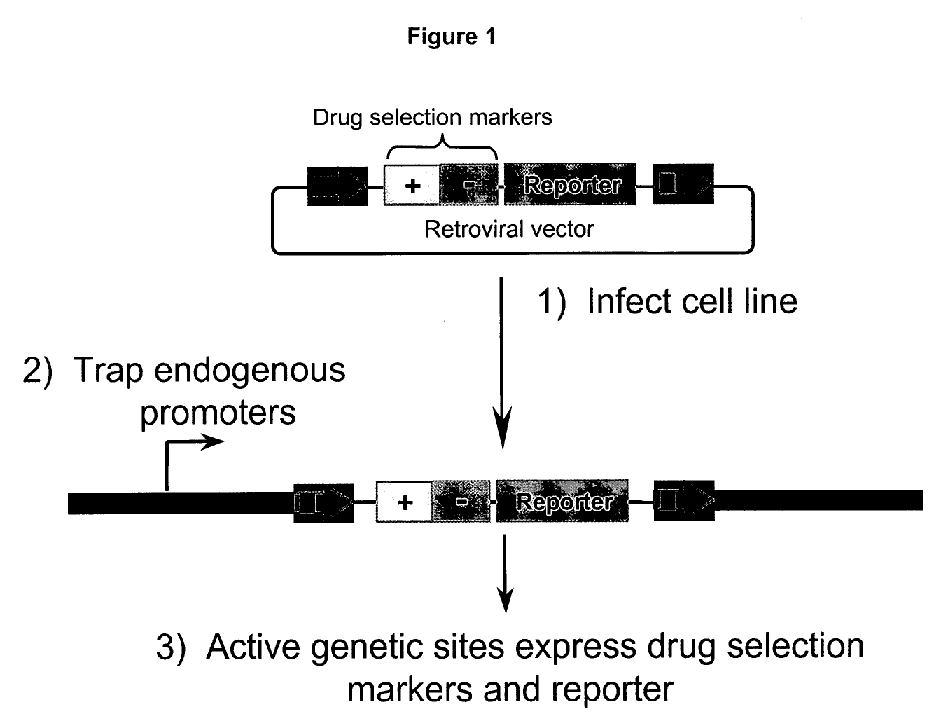

FIG. 1 is a schematic drawing of the Sentinel Line promoter trap system, and its use in identifying regulated genetic sites and in reporting pathway activity. Briefly, the promoter-less selection markers (either positive or negative selection markers, or both) and reporter genes (such as beta-gal) are put in a retroviral vector (or other suitable vectors), which can be used to infect target cells. The randomly inserted retroviral vectors may be so positioned that an active upstream heterologous promoter may initiate the transcription and translation of the selectable markers and reporter gene(s). The expression of such selectable markers and/or reporter genes is indicative of active genetic sites in the particular host cell.

In one exemplary embodiment, the promoter trap vector BV7 was derived from retrovirus vector PQCXIX (BD Biosciences Clontech) by replacing sequence in between packaging signal (Psi⁺) and 3′ LTR with a cassette in an opposite orientation, which contains a splice acceptor sequence derived from mouse engrailed 2 gene (SA/en2), an internal ribosomal entry site (IRES), a LacZ gene, a second IRES, and fusion gene TK:Sh encoding herpes virus thymidine kinase (HSV-tk) and phleomycin followed by a SV40 polyadenylation site. BV7 was constructed by a three-way ligation of three equal molar DNA fragments. Fragment 1 was a 5 kb vector backbone derived from pQCXIX by cutting plasmid DNA extracted from a Dam-bacterial strain with Xho I and Cla I (Dam-bacterial strain was needed here because Cla I is blocked by overlapping Dam methylation). Fragment 2 was a 2.5 kb fragment containing an IRES and a TK:Sh fusion gene derived from plasmid pIREStksh by cutting Dam-plasmid DNA with Cla I and Mlu I. pIREStksh was constructed by cloning TK:Sh fragment from pMODtksh (InvivoGen) into pIRES (BD Biosciences Clontech). Fragment 3 was a 5.8 kb SA/en2-IRES-LacZ fragment derived from plasmid pBSen2IRESLacZ by cutting with BssH II (compatible end to Mlu I) and Xho I. pBSen2IRESLacZ was constructed by cloning IRES fragment from pIRES and LacZ fragment from pMODLacZ (InvivoGen) into plasmid pBSen2.

To prepare virus, packaging cell line 293T was co-transfected with three plasmids BV7, pVSV-G (BD Biosciences Clontech) and pGag-Pol (BD Biosciences Clontech) in equal molar concentrations by using Lipofectamine 2000 (InvitroGen) according to manufacturer's protocol. First virus “soup” (supernatant) was collected 48 hours after transfection, second virus “soup” was collected 24 hours later. Virus particles were pelleted by centrifuging at 25,000 rpm for 2 hours at 4° C. Virus pellets were re-dissolved into DMEM/10% FBS by shaking overnight. Concentrated virus solution was aliquot and used freshly or frozen at −80° C.

### EXAMPLE II

### Sentinel Line Generation

Target cells were plated in 150 mm tissue culture dishes at a density of about 1×10⁶/plate. The following morning cells were infected with 250 μl of Bionaut Virus #7 (BV7) as prepared in Example I, and after 48 hr incubation, 20 μg/ml of phleomycin was added. 4 days later, media was changed to a reduced serum (2% FBS) DMEM to allow the cells to rest. 48 h later, ganciclovir (GCV) (0.4 μM, sigma) was added for 4 days (media was refreshed on day 2). One more round of phleomycin selection followed (20 μg/ml phleomycin for 3 days). Upon completion, media was changed to 20%FBS DMEM to facilitate the outgrowths of the clones. 10 days later, clones were picked and expanded for further analysis and screening.

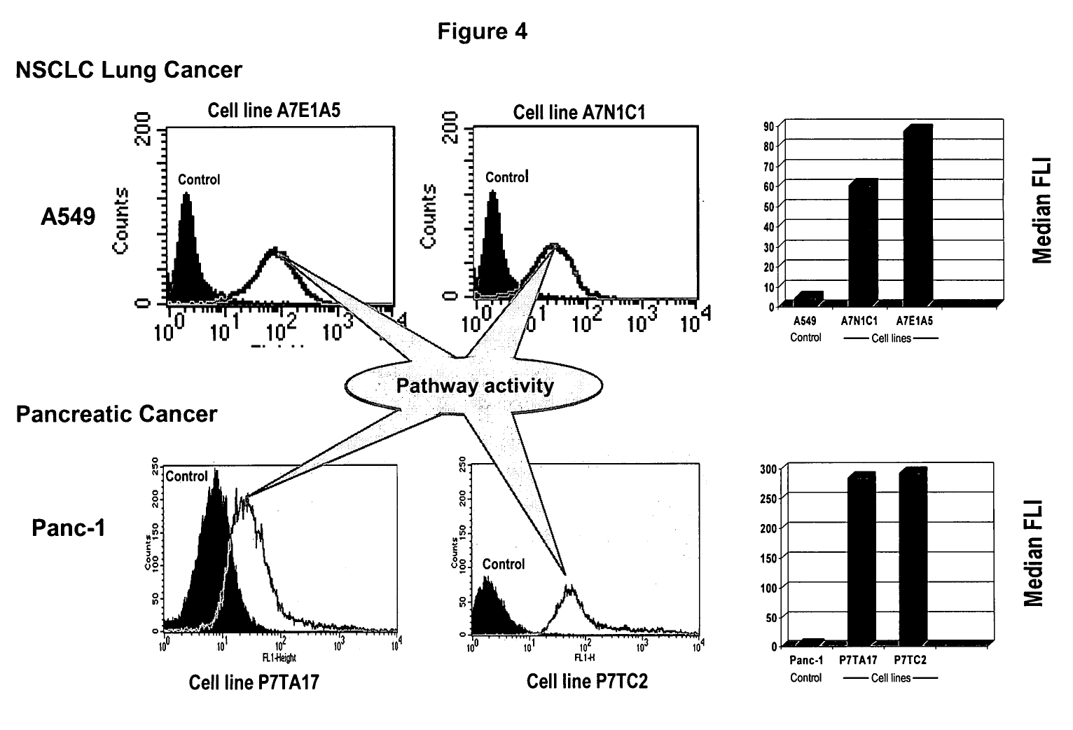

Using this method, several Sentinel Lines were generated to report activity of genetic sites activated by hypoxia pathways (FIG. 4). These Sentinel lines were generated by transfecting A549 (NSCLC lung cancer) and Panc-1 (pancreatic cancer) cell lines with the subject gene-trap vectors containing E. coli LacZ-encoded β-galactosidase (β-gal) as the reporter gene (FIG. 4). The β-gal activity in Sentinel Lines (green) was measured by flow cytometry using a fluorogenic substrate fluoresescein di-beta-D-galactopyranoside (FDG). The autofluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

### EXAMPLE III

### Cell Culture and Hypoxic Conditions

All cell lines can be purchased from ATCC, or obtained from other sources.

A549 (CCL-185) and Panc-1 (CRL-1469) were cultured in Dulbecco's Modified Eagle's Medium (DMEM), Caki-1 (HTB-46) in McCoy's 5a modified medium, Hep3B (HB-8064) in MEM-Eagle medium in humidified atmosphere containing 5% CO₂ at 37° C. Media was supplemented with 10% FBS (Hyclone; SH30070.03), 100 μg/ml penicillin and 50 μg/ml streptomycin (Hyclone).

To induce hypoxia conditions, cells were placed in a Billups-Rothenberg modular incubator chamber and flushed with artificial atmosphere gas mixture (5% CO₂, 1% O₂, and balance N₂). The hypoxia chamber was then placed in a 37° C. incubator. L-mimosine (Sigma, M-0253) was used to induce hypoxia-like HIF1-alpha expression. Proteosome inhibitor, MG132 (Calbiochem, 474791), was used to protect the degradation of HIF1-alpha. Cycloheximide (Sigma, 4859) was used to inhibit new protein synthesis of HIF1-alpha. Catalase (Sigma, C3515) was used to inhibit reactive oxygen species (ROS) production.

### EXAMPLE IV

### Identification of Trapped Genes

Once a Sentinel Line with a desired characteristics was established, it might be helpful to determine the active promoter under which control the markers/reporter genes are expressed. To do so, total RNAs were extracted from cultured Sentinel Line cells by using, for example, RNA-Bee RNA Isolation Reagent (TEL-TEST, Inc.) according to the manufacturer's instructions. Five prime ends of the genes that were disrupted by the trap vector BV7 were amplified by using BD SMART RACE cDNA Amplification Kit (BD Biosciences Clontech) according to the manufacturer's protocol. Briefly, 1 μg total RNA prepared above was reverse-transcribed and extended by using BD PowerScriptase with 5′ CDS primer and BD SMART II Oligo both provided by the kit. PCR amplification were carried out by using BD Advantage 2 Polymerase Mix with Universal Primer A Mix provided by the kit and BV7 specific primer 5′Rsa/ires (gacgcggatcttccgggtaccgagctcc, 28 mer). 5′Rsa/ires located in the junction of SA/en2 and IRES with the first 7 nucleotides matching the last 7 nucleotides of SA/en2 in complementary strand. 5′ RACE products were cloned into the TA cloning vector pCR2.1 (InvitroGen) and sequenced. The sequences of the RACE products were analyzed by using the BLAST program to search for homologous sequences in the database of GenBank. Only those hits which contained the transcript part of SA/en2 were considered as trapped genes.

Using this method, the upstream promoters of several Sentinel Lines generated in Example II were identified (see below). The identity of these trapped genes validate the clinical relevance of these Sentinel Lines™, and can be used as biomarkers and surrogate endpoints in clinical trials.

| Sentinel | | |
|---|---|---|
| Lines | Genetic Sites | Gene Profile |
| A7N1C1 | Essential Antioxidant | Tumor cell-specific gene, over |
| | | expressed in lung tumor cells |
| A7N1C6 | Chr. 3, BAC, map to 3p | novel |
| A7I1C1 | Pyruvate Kinase (PKM 2), | Described biomarker for NSCLC |
| | Chr. 15 | |
| A6E2A4 | 6q14.2-16.1 | Potent angiogenic |
| | | activity |
| A7I1D1 | Chr. 7, BAC | novel |

### EXAMPLE V

### Western Blots

For HIF1-alpha Western blots, Hep3B cells were seeded in growth medium at a density of 7×10⁶ cells per 100 mm dish. Following 24-hour incubation, cells were subjected to hypoxic conditions for 4 hours to induce HIF1-alpha expression together with an agent such as 1 μM BNC1. The cells were harvested and lysed using the Mammalian Cell Lysis kit (Sigma, M-0253). The lysates were centrifuged to clear insoluble debris, and total protein contents were analyzed with BCA protein assay kit (Pierce, 23225). Samples were fractionated on 3-8% Tris-Acetate gel (Invitrogen NUPAGE system) by sodium dodecyl sulfate (SDS)-polyacrylamide gel electropherosis and transferred onto nitrocellulose membrane. HIF1-alpha protein was detected with anti-HIF1-alpha monoclonal antibody (BD Transduction Lab, 610959) at a 1:500 dilution with an overnight incubation at 4° C. in Tris-buffered solution-0.1% Tween 20 (TBST) containing 5% dry non-fat milk. Anti-Beta-actin monoclonal antibody (Abcam, ab6276-100) was used at a 1:5000 dilution with a 30-minute incubation at room temperature. Immunoreactive proteins were detected with stabilized goat-anti mouse HRP conjugated antibody (Pierce, 1858413) at a 1:10,000 dilution. The signal was developed using the West Femto substrate (Pierce, 34095).

We examined the inhibitory effect of BNC1 on HIF-1 alpha synthesis. 24 hours prior to treatment, Hep3B cells were seeded in growth medium. To show that BNC1 inhibits HIF1-alpha expression in a concentration dependent manner, cells were treated with 1 μM BNC1 together with the indicated amount of MG132 under hypoxic conditions for 4 hours. To understand specifically the impact of BNC1 on HIF-1 alpha synthesis, Hep3B cells were treated with MG132 and 1 μM BNC under normoxic conditions for the indicated time points. The observed expression is accounted by protein synthesis.

We examined the role of BNC1 on the degradation rate of HIF-1 alpha. 24 hours prior to treatment, Hep3B cells were seeded in growth medium. The cells were placed in hypoxic conditions for 4 hours for HIF1-alpha accumulation. The protein synthesis inhibitor, cycloheximide (100 μM) together with 1 μM BNC1 were added to the cells and kept in hypoxic conditions for the indicate time points.

To induce HIF1-alpha expression using an iron chelator, L-mimosine was added to Hep3B cells, seeded 24 hours prior, and placed under normoxic conditions for 24 hours.

### EXAMPLE VI

### Sentinel Line Reporter Assays

The expression level of beta-galactosidase gene in sentinel lines was determined by using a fluorescent substrate fluorescein di-B-D-Galactopyranside (FDG, Marker Gene Tech, #M0250) introduced into cells by hypotonic shock. Cleavage by beta-galactosidase results in the production of free fluorescein, which is unable to cross the plasma membrane and is trapped inside the beta-gal positive cells. Briefly, the cells to be analyzed are trypsinized, and resuspended in PBS containing 2 mM FDG (diluted from a 10 mM stock prepared in 8:1:1 mixture of water: ethanol: DMSO). The cells were then shocked for 4 minutes at 37° C. and transferred to FACS tubes containing cold 1×PBS on ice. Samples were kept on ice for 30 minutes and analyzed by FACS in FL1 channel.

### EXAMPLE VII

### Testing Standard Chemotherapeutic Agents

Sentinel Line cells with beta-galactosidase reporter gene were plated at 1×10⁵ cells/10 cm dish. After overnight incubation, the cells were treated with standard chemotherapeutic agents, such as mitoxantrone (8 nM), paclitaxel (1.5 nM), carboplatin (15 μM), gemcitabine (2.5 nM), in combination with one or more BNC compounds, such as BNC1 (10 nM), BNC2 (2 μM), BNC3 (100 μM) and BNC4 (10 nM), or a targeted drug, Iressa (4 μM). After 40 hrs, the cells were trypsinized and the expression level of reporter gene was determined by FDG loading.

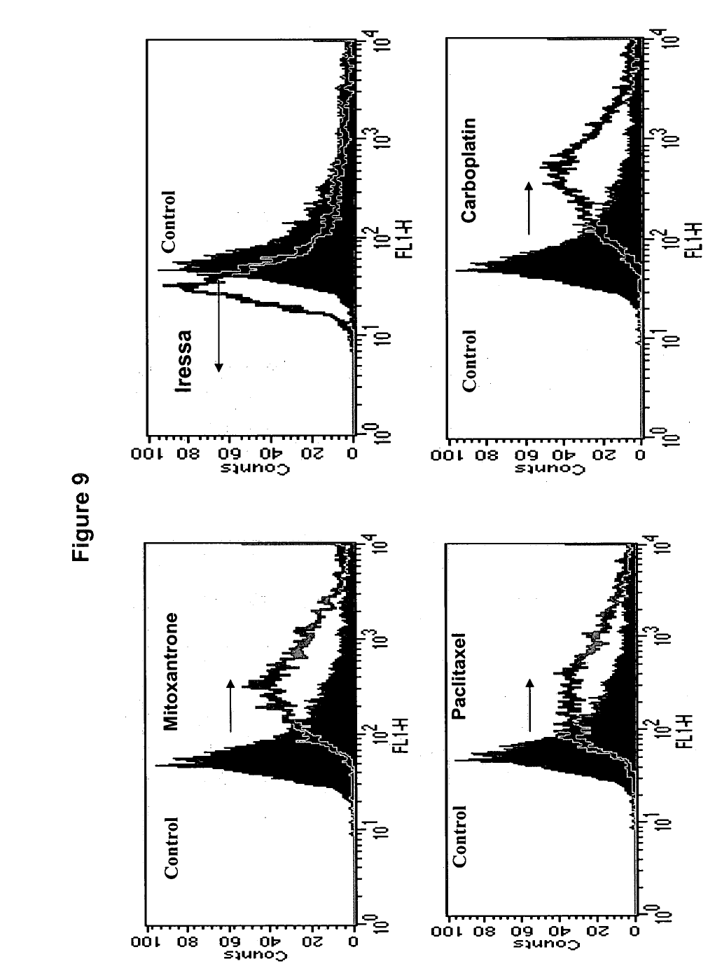

When tested in the Sentinel Lines, mitoxanthrone, paclitaxel, and carboplatin each showed increases in cell death and reporter activity (see FIG. 9). No effect had been expected from the cytotoxic agents because of their nonspecific mechanisms of action (MOA), making their increased reporter activity in HIF-sensitive cell lines surprising. These results provide a previously unexplored link between the development of chemotherapy resistance and induction of the hypoxia response in cells treated with anti-neoplastic agents. Iressa, on the other hand, a known blocker of EGFR-mediated HIF-I induction, showed a reduction in reporter activity when tested. The Sentinel Lines thus provide a means to differentiate between a cytotoxic agent and a targeted drug.

### EXAMPLE VIII

### Pharmacokinetic (PK) Analysis

The following protocol can be used to conduct pharmacokinetic analysis of any compounds of the invention. To illustrate, BNC1 is used as an example.

Nude mice were dosed i.p. with 1, 2, or 4 mg/kg of BNC1. Venous blood samples were collected by cardiac puncture at the following 8 time points: 5 min, 15 min, 30 min, 45 min, 1 hr, 2 hr, 4 hr, 8 hr, and 24 hr. For continuous BNC1 treatment, osmotic pumps (such as Alzet® Model 2002) were implanted s.c. between the shoulder blades of each mouse. Blood was collected at 24 hr, 48 hr and 72 hr. Triplicate samples per dose (i.e. three mice per time point per dose) were collected for this experiment.

Approximately 0.100 mL of plasma was collected from each mouse using lithium heparin as anticoagulant. The blood samples were processed for plasma as individual samples (no pooling). The samples were frozen at −70° C. (±10° C.) and transferred on dry ice for analysis by HPLC.

For PK analysis plasma concentrations for each compound at each dose were analyzed by non-compartmental analysis using the software program WinNonlin®. The area under the concentration vs time curve AUC (0-Tf) from time zero to the time of the final quantifiable sample (Tf) was calculated using the linear trapezoid method. AUC is the area under the plasma drug concentration-time curve and is used for the calculation of other PK parameters. The AUC was extrapolated to infinity (0-Inf) by dividing the last measured concentration by the terminal rate constant (k), which was calculated as the slope of the log-linear terminal portion of the plasma concentrations curve using linear regression. The terminal phase half-life (t_{1/2}) was calculated as 0.693/k and systemic clearance (C1) was calculated as the dose(mg/kg)/AUC(Inf). The volume of distribution at steady-state (Vss) was calculated from the formula:
Vₛₛ=dose(AUMC)/(AUC)²

where A UMC is the area under the first moment curve (concentration multiplied by time versus time plot) and AUC is the area under the concentration versus time curve. The observed maximum plasma concentration (Cₘₐₓ) was obtained by inspection of the concentration curve, and Tₘₐₓ is the time at when the maximum concentration occurred.

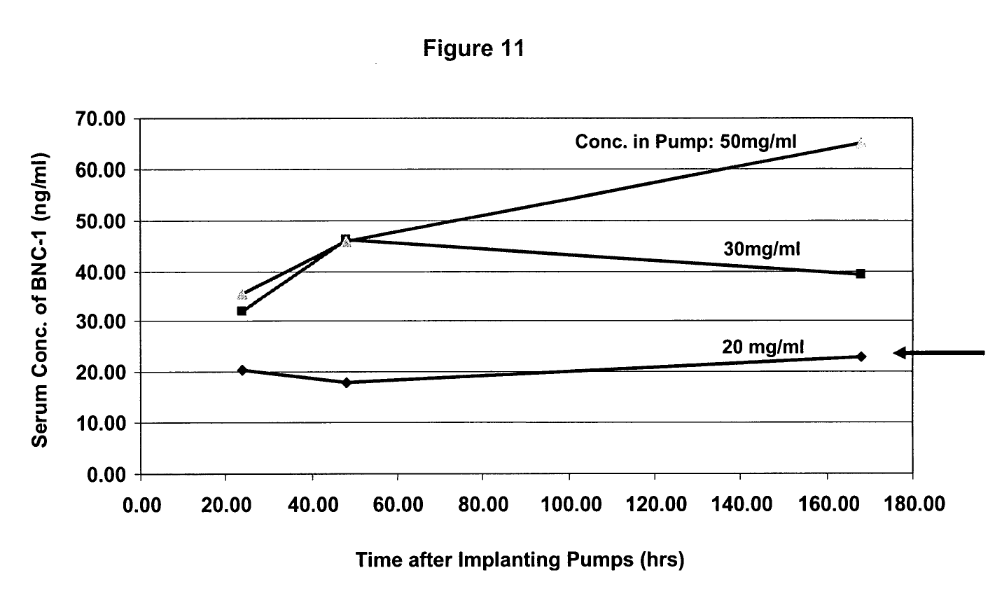

FIG. 11 shows the result of a representative pharmacokinetic analysis of BNC1 delivered by osmotic pumps. Osmotic pumps (Model 2002, Alzet Inc) containing 200 μl of BNC1 at 50, 30 or 20 mg/ml in 50% DMSO were implanted subcutaneously into nude mice. Mice were sacrificed after 24, 48 or 168 hrs, and plasma was extracted and analyzed for BNC1 by LC-MS. The values shown are average of 3 animals per point.

### EXAMPLE IX

### Human Tumor Xenograft Models

Female nude mice (nu/nu) between 5 and 6 weeks of age weighing approximately 20 g were implanted subcutaneously (s.c.) by trocar with fragments of human tumors harvested from s.c. grown tumors in nude mice hosts. When the tumors were approximately 60-75 mg in size (about 10-15 days following inoculation), the animals were pair-matched into treatment and control groups. Each group contains 8-10 mice, each of which was ear tagged and followed throughout the experiment.

The administration of drugs or controls began the day the animals were pair-matched (Day 1). Pumps (Alzet® Model 2002) with a flow rate of 0.5 μl/hr were implanted s.c. between the shoulder blades of each mice. Mice were weighed and tumor measurements were obtained using calipers twice weekly, starting Day 1. These tumor measurements were converted to mg tumor weight by standard formula, (W²×L)/2. The experiment is terminated when the control group tumor size reached an average of about 1 gram. Upon termination, the mice were weighed, sacrificed and their tumors excised. The tumors were weighed and the mean tumor weight per group was calculated. The change in mean treated tumor weight/the change in mean control tumor weight×100 (dT/dC) is subtracted from 100% to give the tumor growth inhibition (TGI) for each group.

### EXAMPLE X

### Cardiac Glycoside Compounds Inhibits HIF-1α Expression

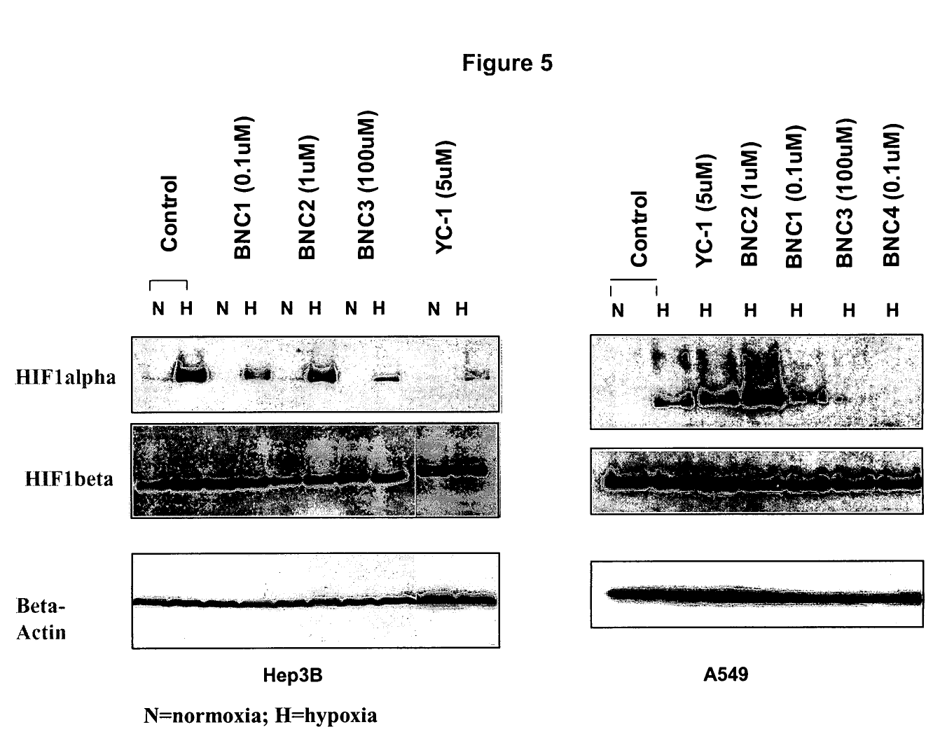

Cardiac glycoside compounds of the invention targets and inhibits the expression of HIF 1α based on Western blot analysis using antibodies specific for HIF1α (FIG. 5).

Hep3B or A549 cells were cultured in complete growth medium for 24 hours and treated for 4 hrs with the indicated cardiac glycoside compounds or controls under normoxia (N) or hypoxia (H) conditions. The cells were lysed and proteins were resolved by SDS-PAGE and transferred to a nylon membrane. The membrane was immunoblotted with anti-HIF1α and anti-HIF1β MAb, and anti-beta-actin antibodies.

In Hep3B cells, various effective concentrations of BNC compounds (cardiac glycoside compounds of the invention) inhibits the expression of HIF-1α, but not HIF-1β. The basic observation is the same, with the exception of BNC2 at 1 μM concentration.

To study the mechanism of HIF-1α inhibition by the subject cardiac glycoside compounds, Hep3B cells were exposed to normoxia or hypoxia for 4 hrs in the presence or absence of: an antioxidant enzyme and reactive oxygen species (ROS) scavenger catalase (1000 U), prolyl-hydroxylase (PHD) inhibitor L-mimosine, or proteasome inhibitor MG132 as indicated. HIF1α and β-actin protein level was determined by western blotting.

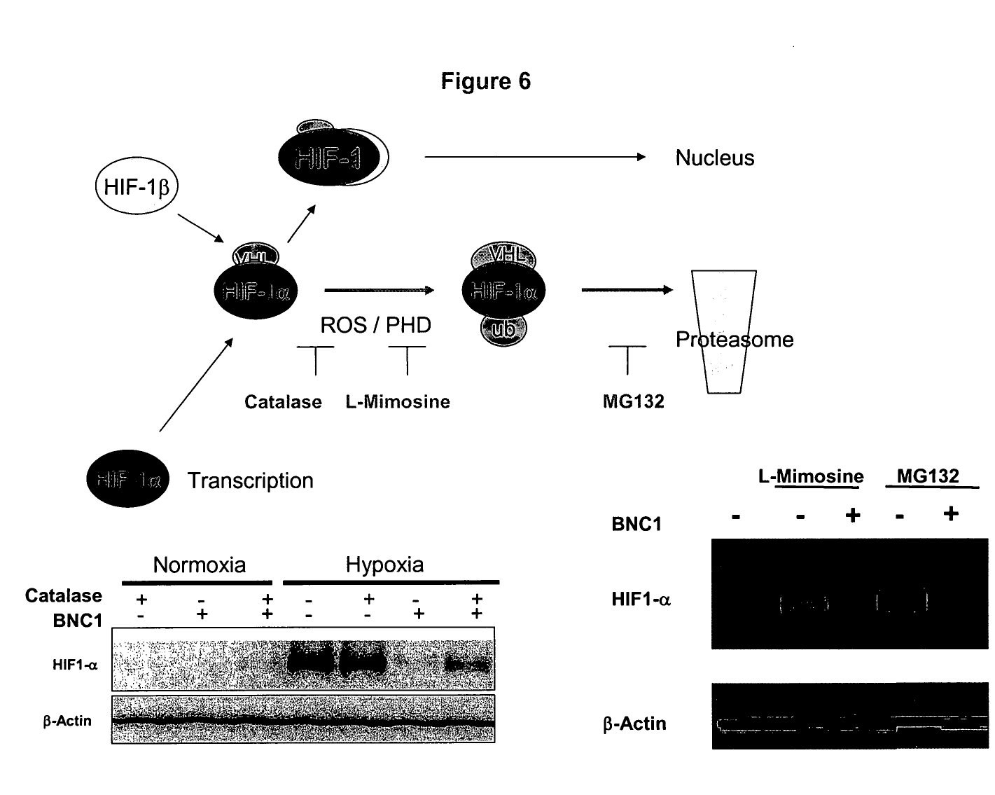

FIG. 6 indicates that the cardiac glycoside compound BNC1 may inhibits steady state HIF-1α level through inhibiting the synthesis of HIF-1α.

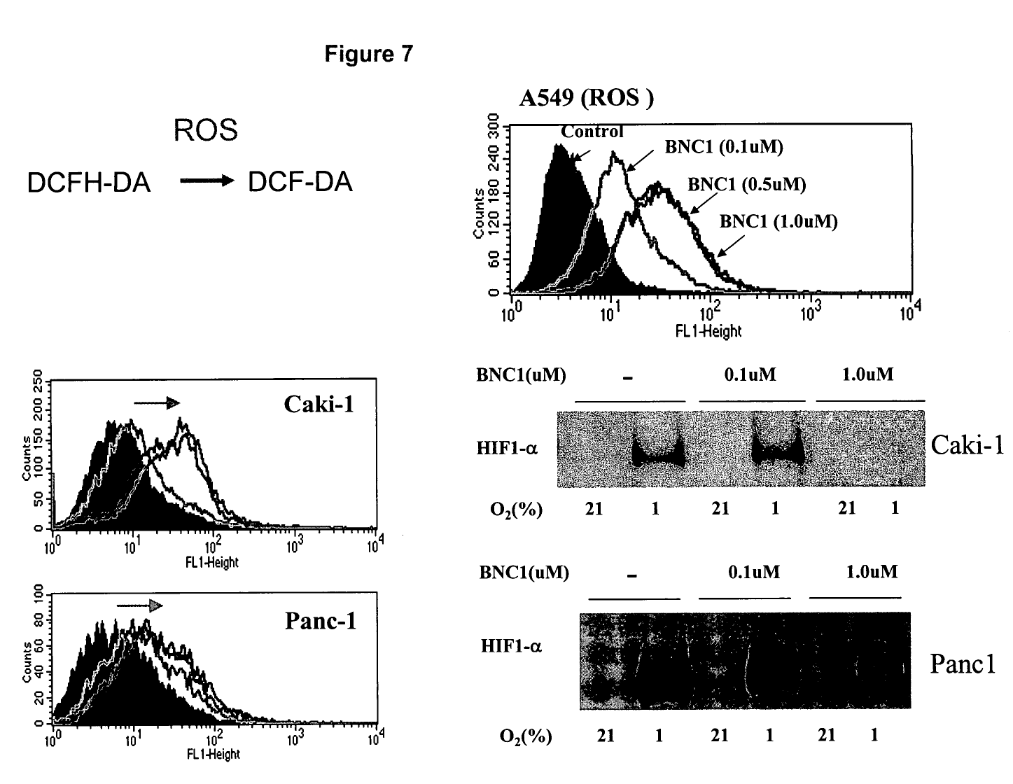

In a related study, tumor cell line A549(ROS) were incubated in normoxia in the absence (control) or presence of different amounts of BNC1 for 4 hrs. Thirty minutes prior to the termination of incubation period, 2,7-dichlorofluorescin diacetate (CFH-DA, 10 mM) was added to the cells and incubated for the last 30 min at 37° C. The ROS levels were determined by FACS analysis. HIF1α protein accumulation in Caki-1 and Panc-1 cells was determined by western blotting after incubating the cells for 4 hrs in normoxia (21% O₂) or hypoxia (1% O₂) in the presence or absence of BNC1. FIG. 7 indicates that BNC1 induces ROS production (at least as evidenced by the A549(ROS) Sentinel Lines), and inhibits HIF1α protein accumulation in the test cells.

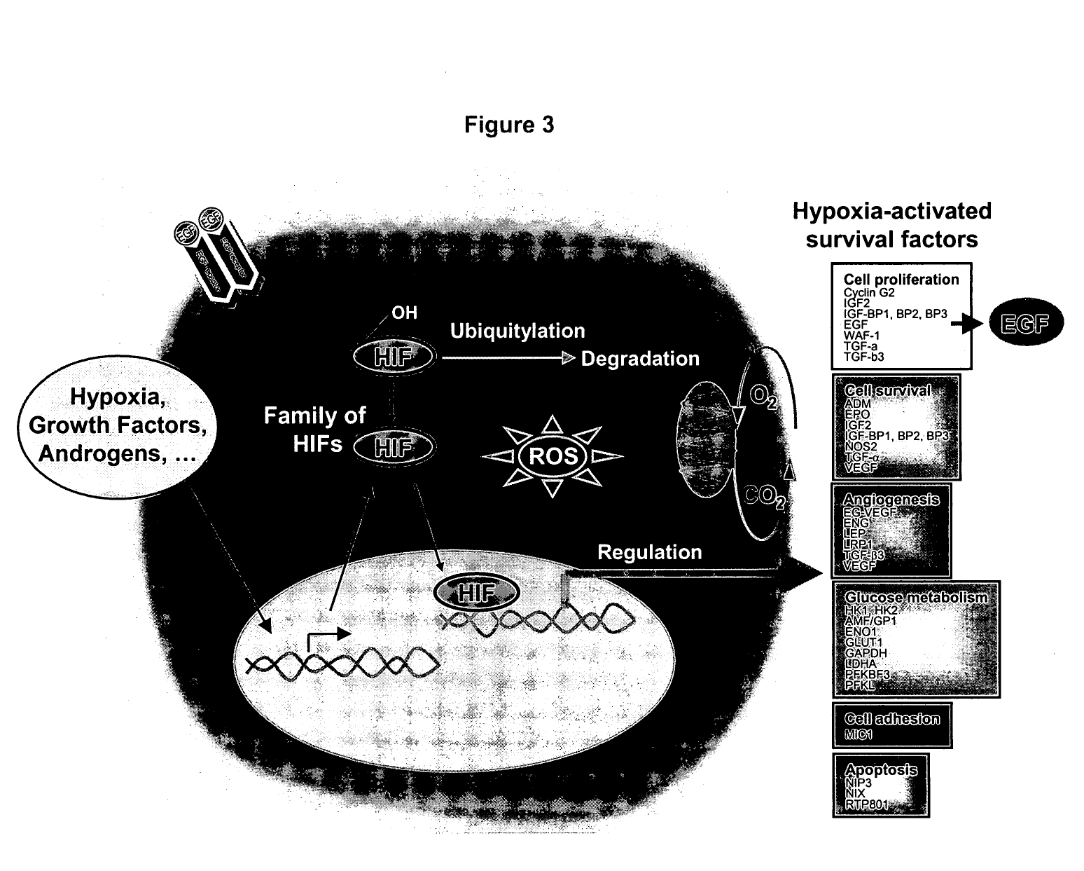

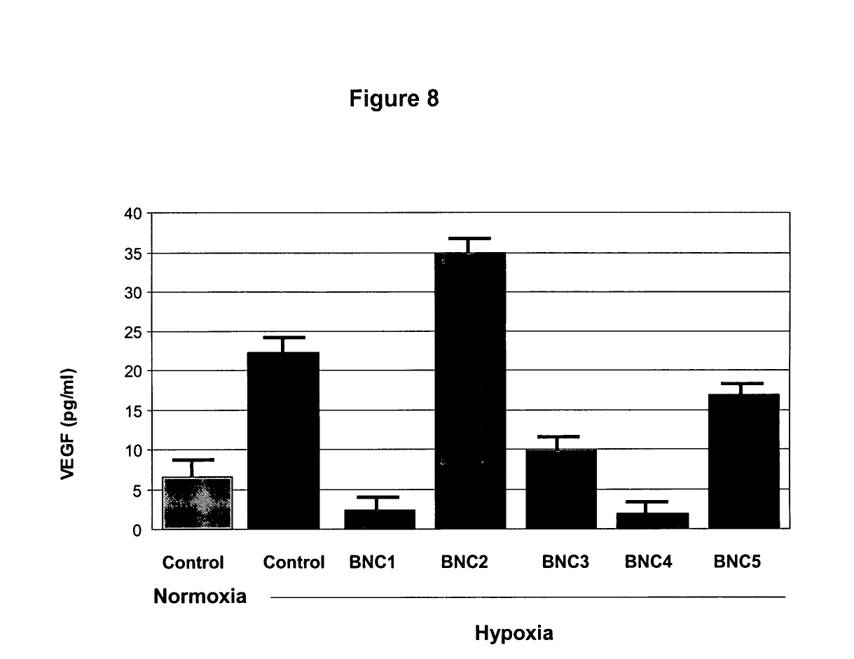

FIG. 8 also demonstrates that the cardiac glycoside compounds BNC1 and BNC4 directly or indirectly inhibits in tumor cells the secretion of the angiogenesis factor VEGF, which is a downstream effector of HIF-1α (see FIG. 3). In contrast, other non-cardiac glycoside compounds, BNC2, BNC3 and BNC5, do not inhibit, and in fact greatly enhances VEGF secretion.

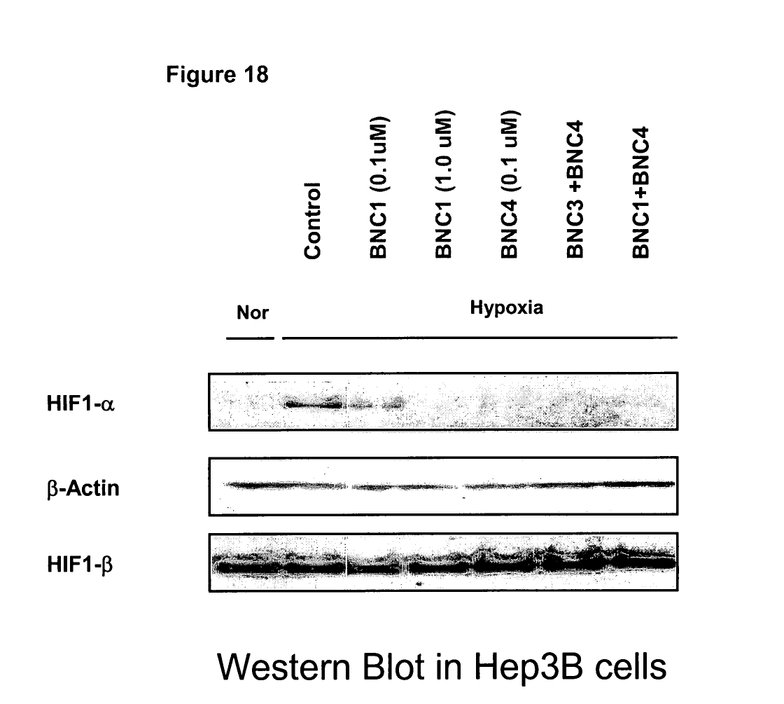

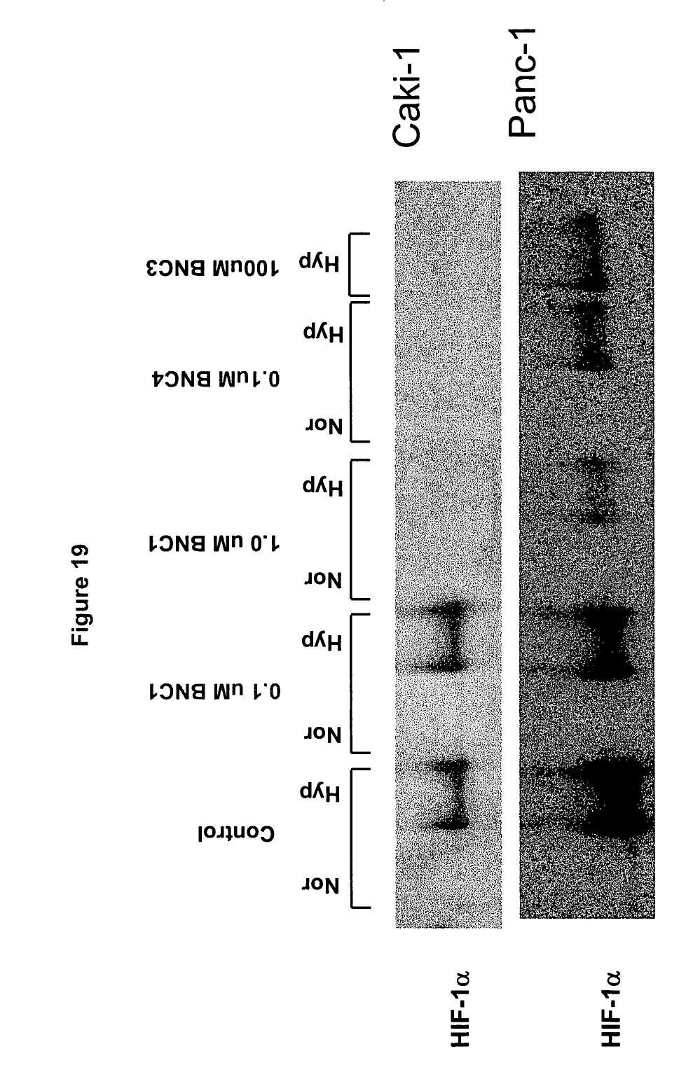

FIGS. 18 and 19 compared the ability of BNC1 and BNC4 in inhibiting hypoxia-mediated HIF1α induction in human tumor cells. The figures show result of immunoblotting for HIF-1α, HIF-1β and β-actin (control) expression, in Hep3B, Caki-1 or Panc-1 cells treated with BNC1 or BNC4 under hypoxia. The results indicate that BNC4 is even more potent (about 10-times more potent) than BNC1 in inhibiting HIF-1α expression.

### EXAMPLE XI

### Neutralization of Gemcitabine-Induced Stress Response as Measured in A549 Sentinel Line

The cardiac glycoside compounds of the invention were found to be able to neutralize Gemcitabine-induced stress response in tumor cells, as measured in A549 Sentinal Lines.

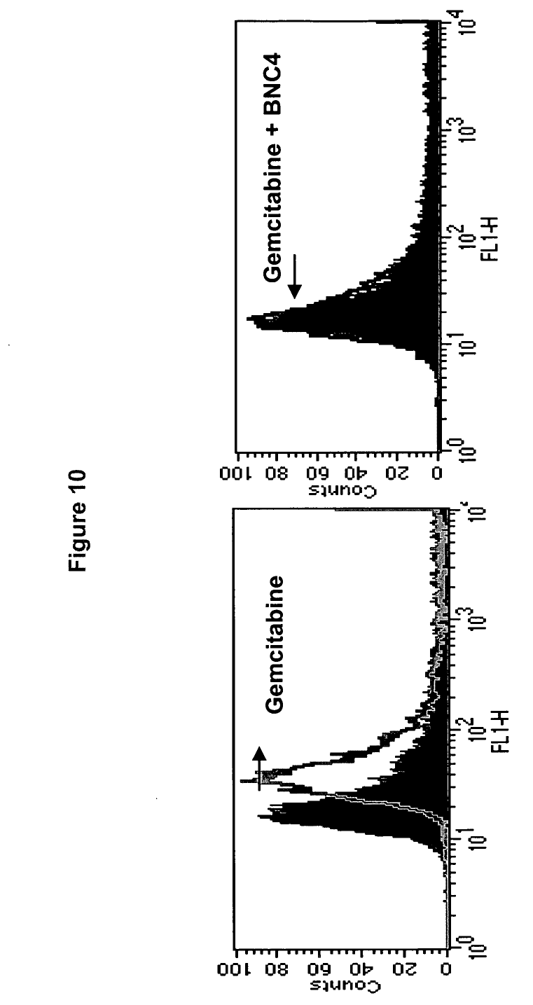

In experiments of FIG. 10, the A549 sentinel line was incubated with Gemcitabine in the presence or absence of indicated Bionaut compounds (including the cardiac glycoside compound BNC4) for 40 hrs. The reporter activity was measured by FACS analysis.

It is evident that at least BNC4 can significantly shift the reporter activity to the left, such that Gemcitabine and BNC4-treated cells had the same reporter activity as that of the control cells. In contrast, cells treated with only Gemcitabine showed elevated reporter activity.

### EXAMPLE XII

### Effect of BNC1 Alone or in Combination with Standard Chemotherapy on Growth of Xenografted Human Pancreatic Tumors in Nude Mice

To test the ability of BNC1 to inhibit xenographic tumor growth in nude mice, either along or in combination with a standard chemotherapeutic agent, such as Gemcitabine, Panc-1 tumors were injected subcutaneously (sc) into the flanks of male nude mice. After the tumors reached 80 mg in size, osmotic pumps (model 2002, Alzet Inc., flow rate 0.5 μl/hr) containing 20 mg/ml of BNC1 were implanted sc on the opposite sides of the mice. The control animals received pumps containing vehicle (50% DMSO in DMEM). The mice treated with standard chemotherapy agent received intra-peritoneal injections of Gemcitabine at 40 mg/kg every 3 days for 4 treatments (q3d×4). Each data point represent average tumor weight (n=8) and error bars indicate SEM.

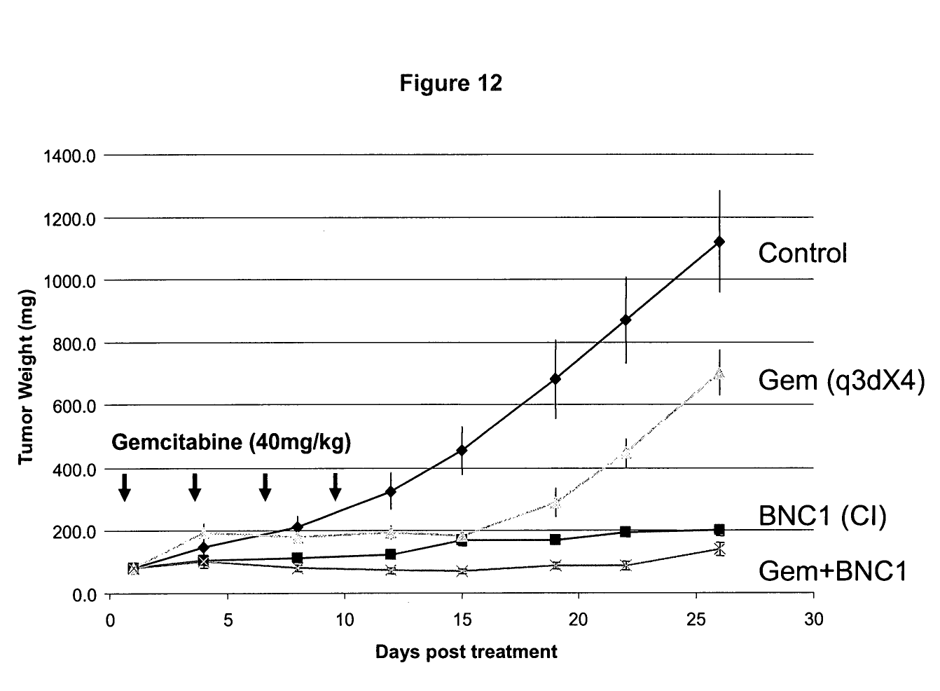

FIG. 12 indicates that, at the dosage tested, BNC1 alone can significantly reduce tumor growth in this model. This anti-tumor activity is additive when BNC1 is co-administered with a standard chemotherapeutic agent Gemcitabine. Results of the experiment is listed below:

| | | Final Tumor | | |
|---|---|---|---|---|
| | | weight Day | | |
| Group (Animal No.) | Dose/Route | 25 (Mean) | SEM | % TGI |
| Control (8) | Vehicle/i.v. | 1120.2 | 161.7 | — |
| BNC1 (8) | 20 mg/ml; | 200 | 17.9 | 82.15 |
| | s.c.; C.I. | | | |
| Gemcitabine (8) | 40 mg/kg; | 701.3 | 72.9 | 37.40 |
| | q3d × 4 | | | |
| BNC1 + Gem (8) | Combine both | 140.8 | 21.1 | 87.43 |

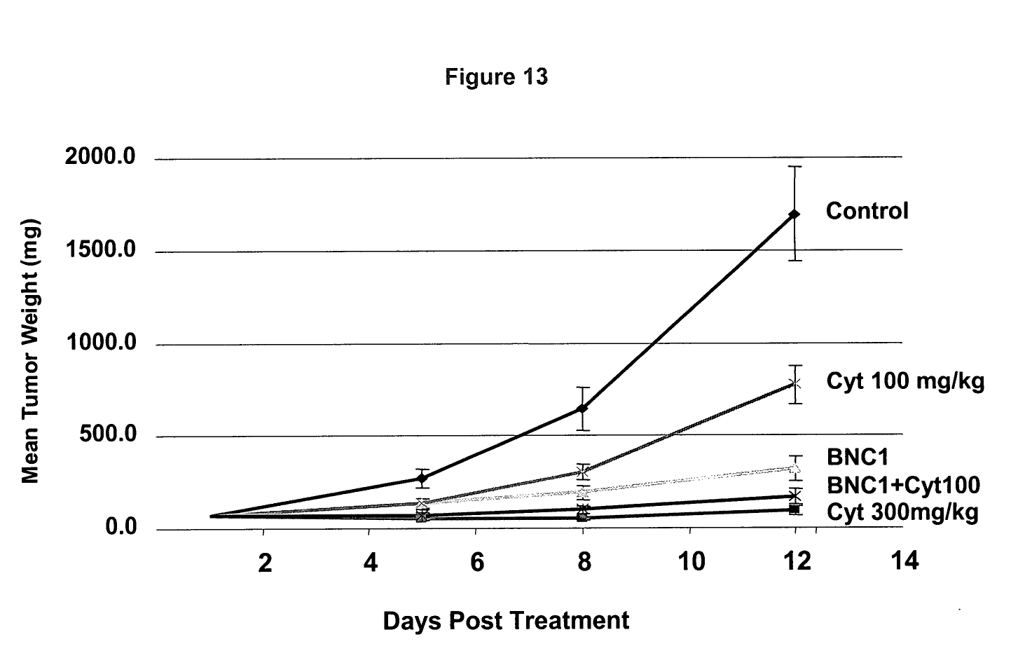

Similarly, in the experiment of FIG. 13, BNC1 (20 mg/ml) was delivered by sc osmotic pumps (model 2002, Alzet Inc.) at 0.5 μl/hr throughout the study. Cytoxan (q1d×1) was injected at 100 mg/kg (Cyt 100) or 300 mg/kg (Cyt 300). The results again shows that BNC1 is a potent anti-tumor agent when used alone, and its effect is additive with other chemotherapeutic agents such as Cytoxan. The result of this study is listed in the table below:

| | | Final Tumor | | |
|---|---|---|---|---|
| Group | | weight Day | | |
| (Animal No.) | Dose/Route | 22 (Mean) | SEM | % TGI |
| Control (10) | Vehicle/i.v. | 1697.6 | 255.8 | — |
| BNC1 (10) | 20 mg/ml; | 314.9 | 67.6 | 81.45 |
| | s.c.; C.I. | | | |
| Cytoxan300 (10) | 300 mg/ml; | 93.7 | 24.2 | 94.48 |
| | ip; qd × 1 | | | |
| Cytoxan100 (10) | 100 mg/ml; | 769 | 103.2 | 54.70 |
| | ip; qd × 2 | | | |
| BNC1 + | Combine | 167 | 39.2 | 90.16 |
| Cytoxan100 (10) | both | | | |

In yet another experiment, the anti-tumor activity of BNC1 alone or in combination with Carboplatin was tested in A549 human non-small-cell-lung carcinoma. In this experiment, BNC1 (20 mg/ml) was delivered by sc osmotic pumps (model 2002, Alzet Inc.) at 0.5 μl/hr throughout the study. Carboplatin (q1d×1) was injected at 100 mg/kg (Carb).

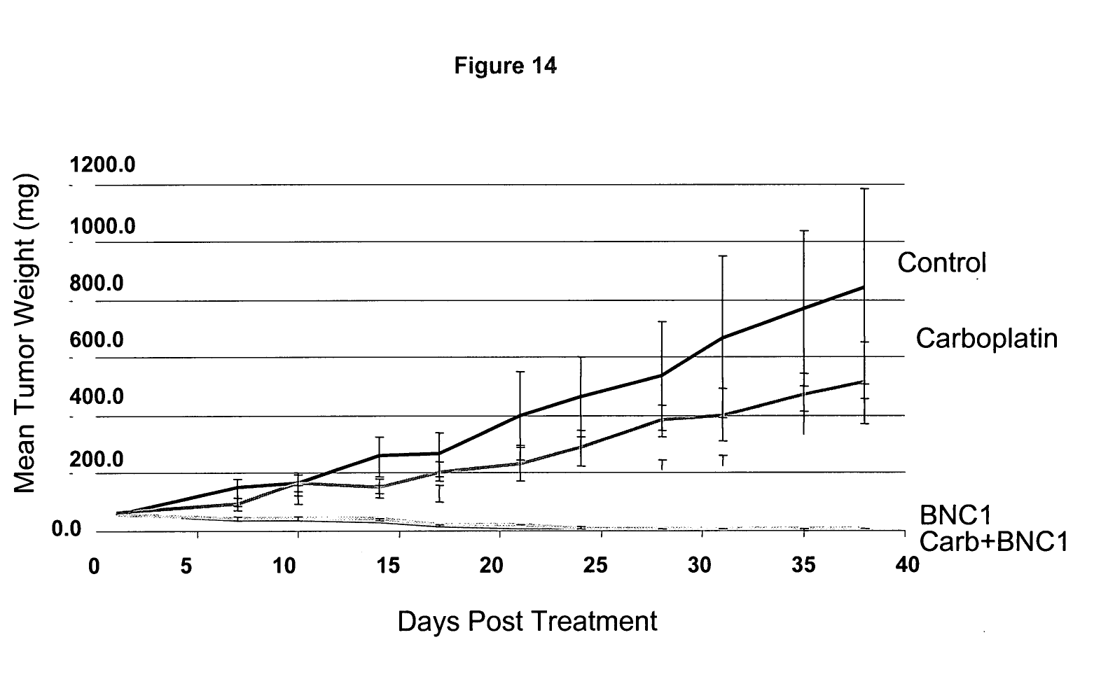

FIG. 14 confirms that either BNC1 alone or in combination with Carboplatin has potent anti-tumor activity in this tumor model. The detailed results of the experiment is listed in the table below:

| | | % Weight | Final Tumor | | |
|---|---|---|---|---|---|
| Group | | Change at | weight Day | | |
| (Animal No.) | Dose/Route | Day 38 | 38 (Mean) | SEM | % TGI |
| Control (8) | Vehicle/i.v. | 21% | 842.6 | 278.1 | — |
| BNC1 (8) | 20 mg/ml; | 21% | 0.0 | 0.0 | 100.00 |
| | s.c.; C.I. | | | | |
| Carboplatin | 100 mg/kg; | 16% | 509.75 | 90.3 | 39.50 |
| (8) | ip; qd × 1 | | | | |
| BNC1 + | Combine | 0% | 0.0 | 0.0 | 100.00 |
| Carb (8) | both | | | | |

Notably, in both the BNC1 and BNC1/Carb treatment group, none of the experimental animals showed any signs of tumor at the end of the experiment, while all 8 experimental animals in control and Carb only treatment groups developed tumors of significant sizes.

Thus the cardiac glycoside compounds of the invention (e.g. BNC1) either alone or in combination with many commonly used chemotherapeutic agents (e.g. Carboplatin, Gem, Cytoxan, etc.) has potent anti-tumor activities in various xenographic animal models of pancreatic cancer, renal cancer, hepatic, and non-small cell lung carcinoma.

### EXAMPLE XIII

### Determining Minimum Effective Dose

Given the additive effect of the subject cardiac glycosides with the traditional chemotherapeutic agents, it is desirable to explore the minimal effective doses of the subject cardiac glycosides for use in conjoint therapy with the other anti-tumor agents.

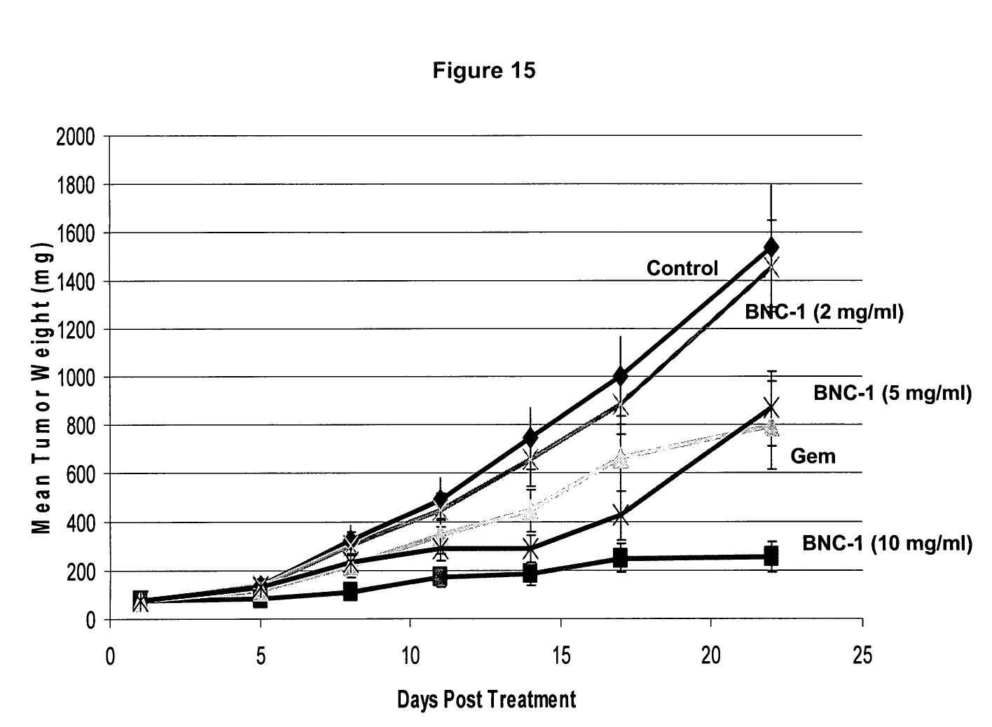

FIG. 15 shows the titration of the exemplary cardiac glycoside BNC1 to determine its minimum effective dose, effective against Panc-1 human pancreatic xenograft in nude mice. BNC1 (sc, osmotic pumps) was first tested at 10, 5 and 2 mg/ml. Gem was also included in the experiment as a comparison.

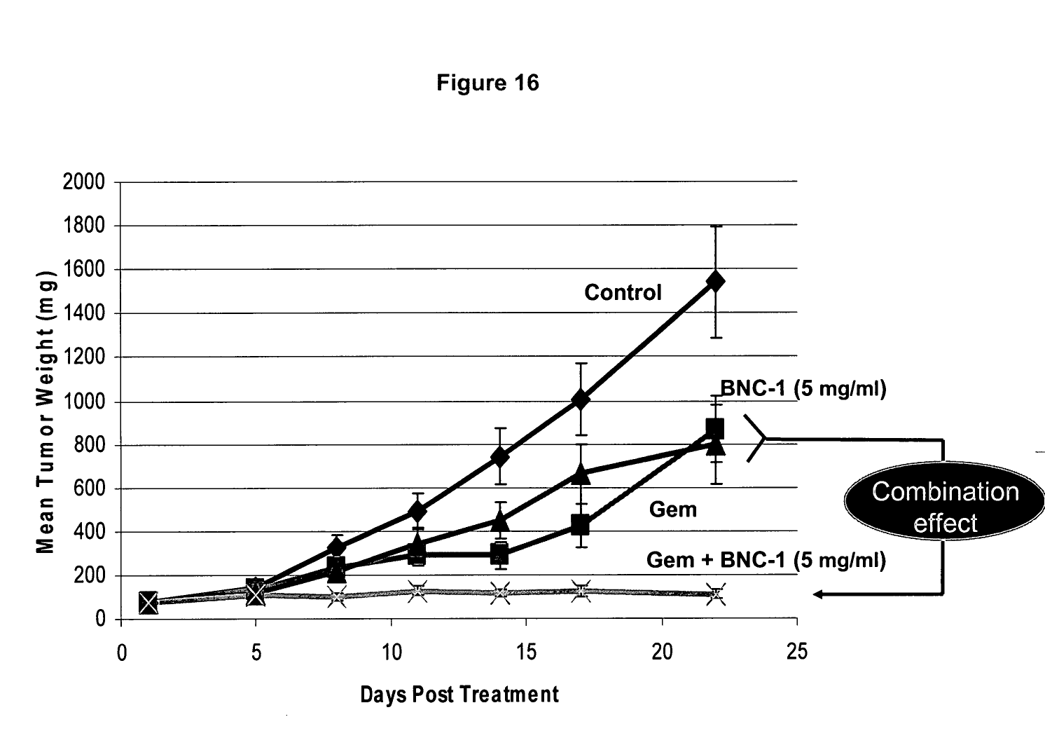

FIG. 16 shows that combination therapy using both Gem and BNC1 produces a combination effect, such that sub-optimal doses of both Gem and BNC1, when used together, produce the maximal effect only achieved by higher doses of individual agents alone.

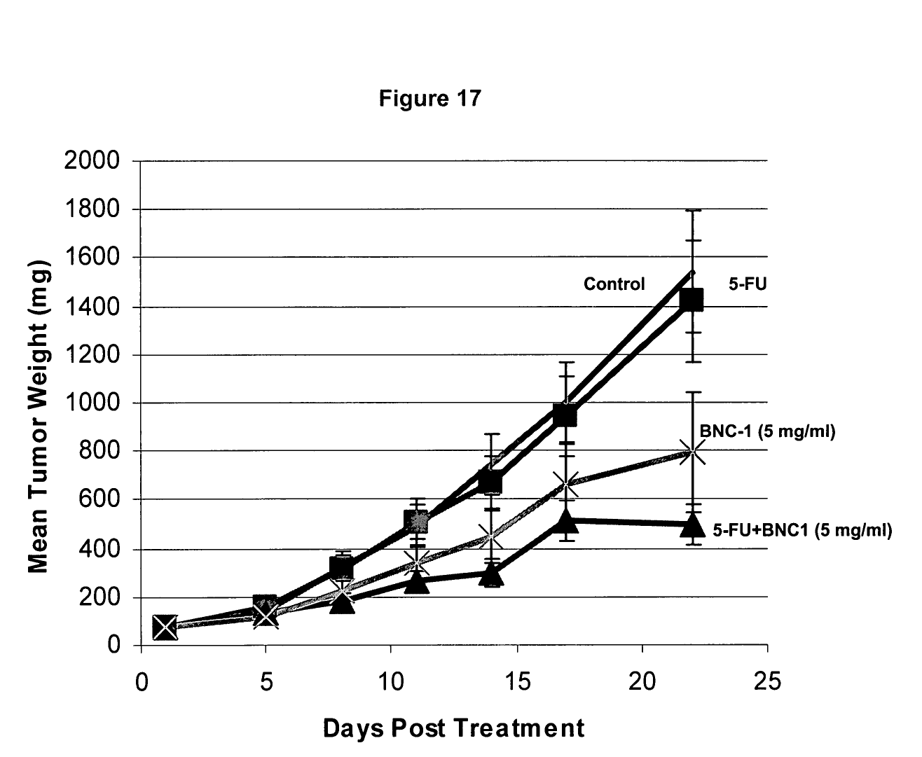

A similar experiment was conducted using BNC1 and 5-FU, and the same combination effect was seen (see FIG. 17).

Similar results are also obtained for other compounds (e.g. BNC2) that are not cardiac glycoside compounds (data not shown).

### EXAMPLE XIV

### BNC4 Inhibits HIF-1α Induced Under Normoxia by PHD Inhibitor

As an attempt to study the mechanism of BNC4 inhibition of HIF-1α, we tested the ability of BNC1 and BNC4 to inhibit HIF-1α expression induced by a PHD inhibitor, L-mimosone (see FIG. 6), under normoxia condition.

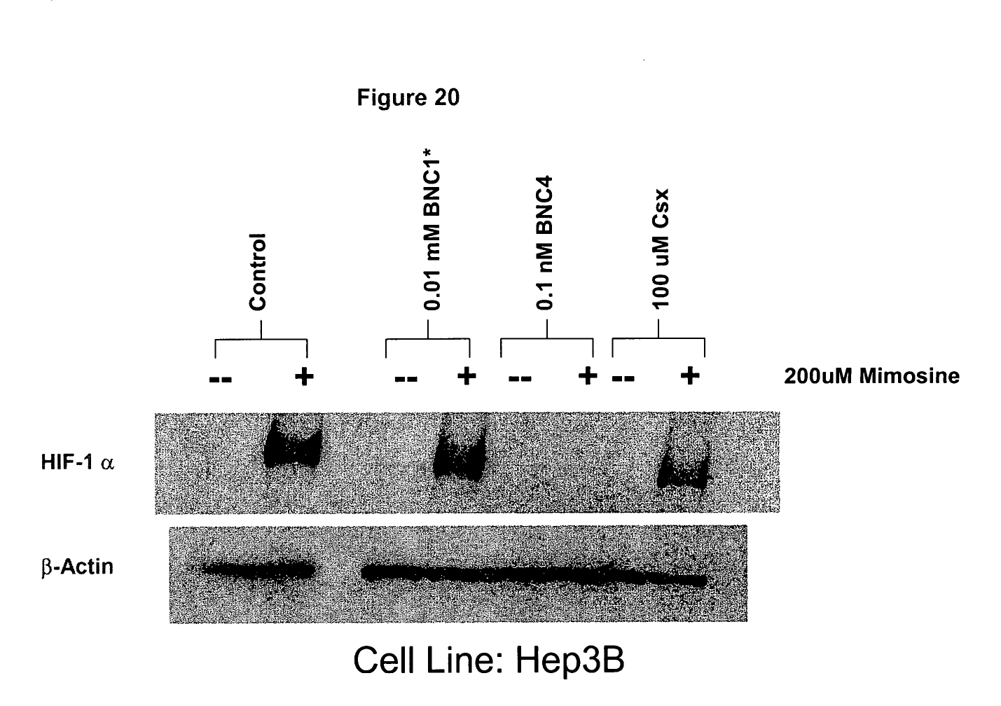

In the experiment represented in FIG. 20, Hep3B cells were grown under normoxia, but were also treated as indicated with 200 μM L-mimosone for 18 h in the presence or absence of BNC1 or BNC4. Abundance of HIF1α and β-actin was determined by western blotting.

The results indicate that L-mimosone induced HIF-1α accumulation under normoxia condition, and addition of BNC4 eliminated HIF-1α accumulation by L-mimosone. At the low concentration tested, BNC1 did not appear to have an effect on HIF-1α accumulation in this experiment. While not wishing to be bound by any particular theory, the fact that BNC4 can inhibit HIF-1α induced under normoxia by PHD inhibitor indicates that the site of action by BNC4 probably lies down stream of prolyl-hydroxylation.

All publications and patents mentioned herein are hereby incorporated by reference in their entirety as if each individual publication or patent was specifically and individually indicated to be incorporated by reference. In case of conflict, the present application, including any definitions herein, will control.

Equivalents:

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A pharmaceutical formulation comprising a Na⁺/K⁺-ATPase inhibitor, either alone or in combination with an anti-angiogenesis agent, formulated in a pharmaceutically acceptable excipient and suitable for use in human patients to reduce angiogenesis.

2. A kit for treating a patient having excessive or undesirable angiogenesis, comprising a Na⁺/K⁺-ATPase inhibitor, either alone or in combination with an anti-angiogenesis agent, formulated in premeasured doses for conjoint administration to said patient.

3. A method for treating a patient having excessive or undesirable angiogenesis, comprising administering to the patient an effective amount of a Na⁺/K⁺-ATPase inhibitor, either alone or in combination with an anti-angiogenesis agent.

4. A method for promoting treatment of patients having excessive or undesirable angiogenesis, comprising packaging, labeling and/or marketing a Na⁺/K⁺-ATPase inhibitor, either alone or in combination with an anti-angiogenesis agent, for use in therapy or conjoint therapy for treating said patients.

5. A method for promoting treatment of patients having excessive or undesirable angiogenesis, comprising packaging, labeling and/or marketing an anti-angiogenesis agent to be used in conjoint therapy with a Na⁺/K⁺-ATPase inhibitor for treating the patients.

6. The pharmaceutical formulation of claim 1, wherein the angiogenesis is induced by hypoxia, or occurs in a non-pathogenic or non-neoplastic condition.

7. The pharmaceutical formulation of claim 1, wherein the Na⁺/K⁺-ATPase inhibitor is a cardiac glycoside agonist.

8. The pharmaceutical formulation of claim 7, wherein the cardiac glycoside agonist, when in combination with the anti-angiogenesis agent,
(1) has an IC₅₀ for inhibiting proliferation or function of one or more different endothelial cell lines that is at least 2 fold less relative to the IC₅₀ of the cardiac glycoside agonist alone, or,
(2) has an EC₅₀ for treating the angiogenesis disorder that is at least 2 fold less relative to the EC₅₀ of the cardiac glycoside agonist alone.

9. The pharmaceutical formulation of claim 7, wherein the cardiac glycoside agonist is represented by the general formula: 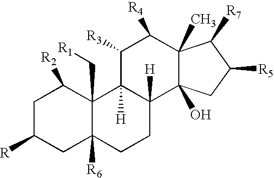
wherein
R represents a glycoside of 1 to 6 sugar residues;
R₁ represents hydrogen, —OH or ═O;
R₂, R₃, R₄, R₅, and R₆ each independently represents hydrogen or —OH; and
R₇ represents 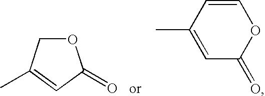
which cardiac glycoside agonist has an IC₅₀ for inhibiting proliferation or function of one or more different endothelial cell lines of 500 nM or less.

10. The pharmaceutical formulation of claim 7, wherein the cardiac glycoside agonist comprises a steroid core with either a pyrone substituent at C17 (the “bufadienolides form”) or a butyrolactone substituent at C17 (the “cardenolide” form).

11. The pharmaceutical formulation of claim 7, wherein the cardiac glycoside agonist is ouabain or proscillaridin.

12. The pharmaceutical formulation of claim 1, wherein the Na⁺/K⁺-ATPase inhibitor inhibits the expression of an angiogenesis factor in said patient.

13. The pharmaceutical formulation of claim 12, wherein the expression of the angiogenesis factor is induced or up-regulated by hypoxia or by HIF-1α.

14. The pharmaceutical formulation of claim 13, wherein the angiogenesis factor is VEGF.

15. The method of claim 3, wherein said excessive or undesirable angiogenesis occurs in lung cancer tissue of the patient, and wherein the Na⁺/K⁺-ATPase inhibitor is at an amount or level sufficient to down-regulate VEGF expression so as to inhibit angiogenesis in said tissue.

16. A method to treat Rheumatoid Arthritis (RA) in a patient, comprising administering to synovial tissue of a bone joint of the patient a composition containing a Na⁺/K⁺-ATPase inhibitor, such as a cardiac glycoside agonist (e.g. ouabain or proscillaridin, etc.), at an amount/level sufficient to down-regulate VEGF expression in synovial tissue and inhibit angiogenesis in the synovial tissue.

17. A method to treat diabetic retinopathy in a patient, comprising administering to a retina of the patient a composition containing a Na⁺/K⁺-ATPase inhibitor, such as a cardiac glycoside agonist (e.g. ouabain or proscillaridin, etc.), at an amount/level sufficient to down-regulate VEGF expression in the retina and inhibit angiogenesis in the retina.

18. A method to treat choroidal neovascularization in a patient, comprising delivering to subretinal space or retinal pigment epithelium of the patient a composition containing a Na⁺/K⁺-ATPase inhibitor, such as a cardiac glycoside agonist (e.g. ouabain or proscillaridin, etc.), at an amount/level sufficient to down-regulate VEGF expression in said tissue and inhibit angiogenesis in the choroidal tissue.

19. The pharmaceutical formulation of claim 1, wherein the anti-angiogenesis agent is:
(1) a VEGF inhibitor selected from: an antibody against VEGF, or a VEGF antigenic epitope; or, (2) a truncated, soluble form of a VEGF receptor selected from: Flt-1, Flk-1/KDR, Flt-4, neuropilin-1 or -2 (NP1 or NP2).

20. A pharmaceutical formulation comprising a Na⁺/K⁺-ATPase inhibitor, either alone or in combination with an agent effective for treating or preventing Alzheimer's Disease (AD), formulated in a pharmaceutically acceptable excipient and suitable for use in human patients to treat or prevent AD.

21. A pharmaceutical formulation comprising an antagonist of a Na⁺/K⁺-ATPase inhibitor, either alone or in combination with an anti-depression agent, formulated in a pharmaceutically acceptable excipient and suitable for use in human patients to reduce depression.

22. A pharmaceutical formulation comprising an antagonist of a Na⁺/K⁺-ATPase inhibitor formulated in a pharmaceutically acceptable excipient and suitable for use in human patients to treat digitalis poinsoning.
